(19) United States
(12) Patent Application Publication
(10) Pub. No.: US 2016/0183534 A1
(43) Pub. Date: Jun. 30, 2016

(54) BACILLUS LICHENIFORMIS RTI184 COMPOSITIONS AND METHODS OF USE FOR BENEFITING PLANT GROWTH

(71) Applicant: FMC Corporation, Philadelphia, PA (US)

(72) Inventors: Safiyh Taghavi, Chapel Hill, NC (US); Daniel van der Lelie, Chapel Hill, NC (US); Jaeheon Lee, Apex, NC (US)

(21) Appl. No.: 14/870,657

(22) Filed: Sep. 30, 2015

Related U.S. Application Data
(60) Provisional application No. 62/171,555, filed on Jun. 5, 2015.
(60) Provisional application No. 62/097,256, filed on Dec. 29, 2014.

(51) Int. Cl. A01N 63/00; A01N 57/12; C12N 1/20; A01N 63/02; C12R 1/10; A01N 53/00; A01N 47/44

(52) U.S. Cl. CPC A01N 63/00; A01N 53/00; A01N 57/12; A01N 47/44; A01N 63/02; C12R 1/10; C12N 1/20

(57) ABSTRACT

Compositions and methods for application to plants are provided for a new strain of Bacillus licheniformis RTI184 having plant growth promoting activity. Compositions and extracts of Bacillus licheniformis strains that include newly identified Fengycin-like and Dehydroxyfengycin-like cyclic lipopeptides designated as “Fengycin MB-Cit” and “Dehydroxyfengycin MB-Cit”, respectively, are also provided. In particular aspects, compositions containing the Bacillus licheniformis strain RTI184 can be applied alone or in combination with chemical agents or other microbial agents to benefit plant growth.

### CROSS REFERENCE TO RELATED APPLICATIONS

This application claims the benefit of U.S. provisional application No. 62/097,256 filed Dec. 29, 2014 and U.S. provisional application No. 62/171,555 filed Jun. 5, 2015, the disclosures of which are each hereby incorporated by reference in their entireties.

### TECHNICAL FIELD

The presently disclosed subject matter relates to compositions comprising an isolated strain of Bacillus licheniformis for application to plant roots, plant seeds, and the soil surrounding plants to benefit plant growth.

### BACKGROUND

A number of microorganisms having beneficial effects on plant growth and health are known to be present in the soil, to live in association with plants specifically in the root zone (Plant Growth Promoting Rhizobacteria “PGPR”), or to reside as endophytes within the plant. Their beneficial plant growth promoting properties include nitrogen fixation, iron chelation, phosphate solubilization, inhibition of non-beneficial microorganisms, resistance to pests, Induced Systemic Resistance (ISR), Systemic Acquired Resistance (SAR), decomposition of plant material in soil to increase useful soil organic matter, and synthesis of phytohormones such as indole-acetic acid (IAA), acetoin and 2,3-butanediol that stimulate plant growth, development and responses to environmental stresses such as drought. In addition, these microorganisms can interfere with a plant's ethylene stress response by breaking down the precursor molecule, 1-aminocyclopropane-1-carboxylate (ACC), thereby stimulating plant growth and slowing fruit ripening. These beneficial microorganisms can improve soil quality, plant growth, yield, and quality of crops. Various microorganisms exhibit biological activity such as to be useful to control plant diseases. Such biopesticides (living organisms and the compounds naturally produced by these organisms) can be safer and more biodegradable than synthetic fertilizers and pesticides.

Fungal phytopathogens, including but not limited to Botrytis spp. (e.g. Botrytis cinerea), Fusarium spp. (e.g. F. oxysporum and F. graminearum), Rhizoctonia spp. (e.g. R. solani), Magnaporthe spp., Mycosphaerella spp., Puccinia spp. (e.g. P. recondita), Phytopthora spp. and Phakopsora spp. (e.g. P. pachyrhizi), are one type of plant pest that can cause servere economic losses in the agricultural and horticultural industries. Chemical agents can be used to control fungal phytopathogens, but the use of chemical agents suffers from disadvantages including high cost, lack of efficacy, emergence of resistant strains of the fungi, and undesirable environmental impacts. In addition, such chemical treatments tend to be indiscriminant and may adversely affect beneficial bacteria, fungi, and arthropods in addition to the plant pathogen at which the treatments are targeted. A second type of plant pest are bacterial pathogens, including but not limited to Erwinia spp. (such as Erwinia chrysanthemi), Pantoea spp. (such as P. citrea), Xanthomonas (e.g. Xanthomonas campestris), Pseudomonas spp. (such as P. syringae) and Ralstonia spp. (such as R. soleacearum) that cause severe economic losses in the agricultural and horticultural industries. Similar to pathogenic fungi, the use of chemical agents to treat these bacterial pathogens suffers from disadvantages. Viruses and virus-like organisms comprise a third type of plant disease-causing agent that is hard to control, but to which bacterial microorganisms can provide resistance in plants via induced systemic resistance (ISR). Thus, microorganisms that can be applied as biofertilizer and/or biopesticide to control pathogenic fungi, viruses, and bacteria are desirable and in high demand to improve agricultural sustainability. A final type of plant pathogen includes plant pathogenic nematodes and insects, which can cause severe damage and loss of plants.

Some members of the species Bacillus have been reported as biocontrol strains, and some have been applied in commercial products (Kloepper, J. W. et al., 2004, Phytopathology Vol. 94, No. 11, 1259-1266). For example, strains currently being used in commercial biocontrol products include: Bacillus licheniformis strain QST2808, used as active ingredient in SONATA and BALLAD-PLUS, produced by BAYER CROP SCIENCE; Bacillus licheniformis strain GB34, used as active ingredient in YIELDSHIELD, produced by BAYER CROP SCIENCE; Bacillus subtilis strain QST713, used as the active ingredient of SERENADE, produced by BAYER CROP SCIENCE; Bacillus subtilis strain GBO3, used as the active ingredient in KODIAK and SYSTEM3, produced by HELENA CHEMICAL COMPANY. Various strains of Bacillus thuringiensis and Bacillus firmus have been applied as biocontrol agents against nematodes and vector insects and these strains serve as the basis of numerous commercially available biocontrol products, including NORTICA and PONCHO-VOTIVO, produced by BAYER CROP SCIENCE. In addition, Bacillus strains currently being used in commercial biostimulant products include: Bacillus amyloliquefaciens strain FZB42 used as the active ingredient in RHIZOVITAL 42, produced by ABiTEP GmbH, as well as various other Bacillus subtilus species that are included as whole cells including their fermentation extract in biostimulant products, such as FULZYME produced by JHBiotech Inc.

The presently disclosed subject matter provides microbial compositions and methods for their use in benefiting plant growth.

### SUMMARY OF THE INVENTION

In one embodiment of the present invention, a composition for benefiting plant growth is provided including a biologically pure culture of Bacillus licheniformis strain RTI184 deposited as ATCC No. PTA-121722, or a mutant thereof having all the identifying characteristics thereof, present in an amount suitable to benefit plant growth.

In one embodiment of the present invention, a coated plant seed is provided, the plant seed coated with a composition comprising spores of a biologically pure culture of Bacillus licheniformis strain RTI184 deposited as ATCC No. PTA-121722, or mutants thereof having all the identifying characteristics thereof, present in an amount suitable to benefit plant growth.

In one embodiment of the present invention, a composition is provided for benefiting plant growth, the composition including a biologically pure culture of Bacillus licheniformis strain RTI184 deposited as ATCC No. PTA-121722, or mutants thereof having all the identifying characteristics thereof; and an insecticide, a herbicide, a fungicide, nematicide, a bacteriocide, a plant growth regulator, a fertilizer, a microbial or a combination thereof present in an amount suitable to benefit plant growth.

In one embodiment of the present invention, a method is provided for benefiting plant growth, the method including delivering a composition including a biologically pure culture of Bacillus licheniformis strain RTI184 deposited as ATCC PTA-121722, or a mutant thereof having all the identifying characteristics thereof to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit plant growth.

In one embodiment of the present invention, a method is provided for benefiting plant growth, the method including: planting a seed of the plant or regenerating vegetative/callus tissue of the plant in a suitable growth medium, wherein the seed has been coated or the vegetative/callus tissue has been inoculated with a composition comprising a biologically pure culture of a Bacillus licheniformis strain RTI184 deposited as ATCC PTA-121722, or a mutant thereof having all the identifying characteristics thereof, wherein growth of the plant from the seed or the vegetative/callus tissue is benefited.

In one embodiment of the present invention, a method is provided for benefiting plant rooting, the method including: dipping a cutting of the plant in a composition and planting it in a suitable growth medium, wherein the composition comprises a biologically pure culture of a Bacillus licheniformis strain RTI184 deposited as ATCC PTA-121722, or a mutant thereof having all the identifying characteristics thereof, in an amount suitable for benefiting plant rooting, wherein root formation and growth of the plant from the cutting is benefited.

In one embodiment of the present invention, a method is provided for benefiting plant growth that includes: delivering a combination of: a first composition comprising a composition comprising a biologically pure culture of a Bacillus licheniformis strain RTI184 deposited as ATCC No. PTA-121722, or mutants thereof having all the identifying characteristics thereof in an amount suitable for benefiting plant growth; and a second composition comprising an insecticide, a herbicide, a fungicide, a nematicide, a bacteriocide, a plant growth regulator, a fertilizer, a microbial, or a combination thereof, in an amount suitable for benefiting plant growth, to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment of the present invention, a method is provided for benefiting plant growth that includes: delivering a composition comprising: a biologically pure culture of Bacillus licheniformis strain RTI184 deposited as ATCC No. PTA-121722, or mutants thereof having all the identifying characteristics thereof, in an amount suitable for benefiting plant growth; and an insecticide, a herbicide, a fungicide, a nematicide, a bacteriocide, a plant growth regulator, a fertilizer, a microbial, or a combination thereof, in an amount suitable for benefiting plant growth to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment of the present invention, a composition is provided for application to a plant, the composition including at least one of an isolated Fengycin MB-Cit compound and an isolated Dehydroxyfengycin MB-Cit compound and optionally one or a combination of additional isolated Fengycin- and Dehydroxyfengycin-like compounds listed in Table VI in an amount suitable to confer one or both of a growth benefit on the plant or protection against a pathogenic infection in the susceptible plant, the Fengycin MB-Cit and Dehydroxyfengycin MB-Cit compounds having the formula:

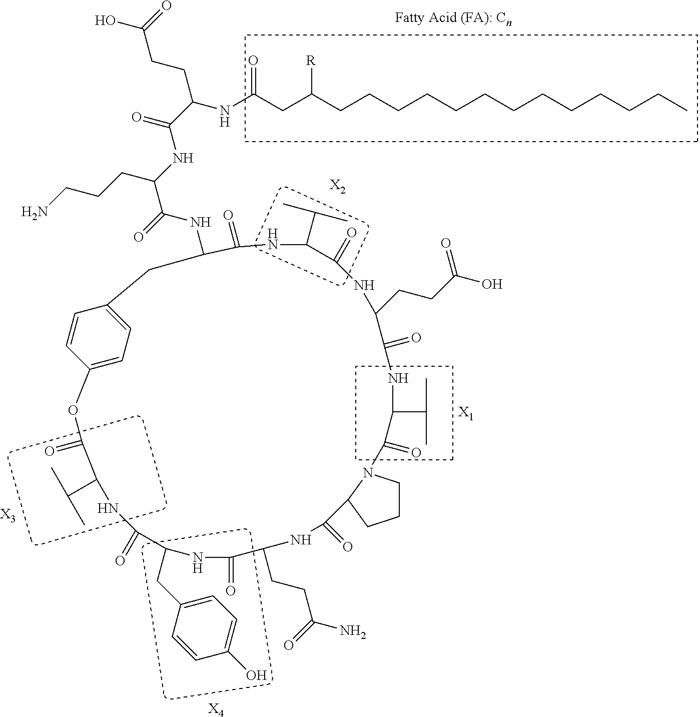

wherein n ranges from 8 to 20, FA is linear, iso, or anteiso, and R is OH, X₁ is Val, X₂ is Thr, X₃ is Met, and X₄ is Cit for Fengycin MB-Cit and wherein n ranges from 8 to 20, FA is linear, iso, or anteiso, R is H, X₁ is Val, X₂ is Thr, X₃ is Met, and X₄ is Citruline for Dehydroxyfengycin MB-Cit.

In one embodiment, an extract is provided of a biologically pure culture of a Bacillus licheniformis strain, the extract including a Fengycin MB-Cit compound and a Dehydroxyfengycin MB-Cit compound and one or a combination of additional Fengycin- and Dehydroxyfengycin-like compounds listed in Table VI.

In one embodiment, an extract is provided of a biologically pure culture of Bacillus licheniformis RTI184 deposited as ATCC No. PTA-121722, the extract including a Fengycin MB-Cit compound and a Dehydroxyfengycin MB-Cit compound and one or a combination of additional Fengycin- and Dehydroxyfengycin-like compounds listed in Table VI.

### BRIEF DESCRIPTION OF THE DRAWINGS

FIG. 1 shows images showing the positive effects on root hair development in soybean seedlings after inoculation of seed with Bacillus licheniformis strain RTI184 at B) 1.04×10⁶ CFU/ml, C) 1.04×10⁵ CFU/ml, and D) 1.04×10⁴ CFU/ml after 7 days of growth as compared to untreated control A) according to one or more embodiments of the present invention.

FIG. 2 shows images showing the positive effects of inoculation of seed with Bacillus licheniformis strain RTI184 on early plant growth in MONEY MAKER tomato according to one or more embodiments of the present invention. The extracted plants after 7 days growth are shown in the figure. A) Control plants; and B) Plants inoculated with RTI184.

FIG. 3 shows images showing the positive effects of inoculation of seed with Bacillus licheniformis strain RTI184 on plant growth in corn according to one or more embodiments of the present invention. A) Plants inoculated with Bacillus licheniformis strain RTI184; and B) Control plants.

FIG. 4 shows images showing the positive effects on growth and vigor in cucumber as a result of addition of Bacillus licheniformis strain RTI184 to PROMIX BX (PREMIER TECH, INC; Quebec, Canada) potting soil limed to pH of 6.5 according to one or more embodiments of the present invention. A) Control cucumber plants in the same soil without addition of Bacillus licheniformis RTI184; and B) Cucumber plants after addition to the soil of 1×10⁷ CFU/g Bacillus licheniformis RTI184 spores.

FIG. 5 shows images showing the positive effects on growth and vigor in tomato as a result of addition of Bacillus licheniformis strain RTI184 to PROMIX BX (PREMIER TECH, INC; Quebec, Canada) potting soil limed to pH of 6.5 according to one or more embodiments of the present invention. A) Tomato plants after addition to soil of 1×10⁷ CFU/g Bacillus licheniformis RTI184 spores; and B) Control tomato plants in the same soil without addition of Bacillus licheniformis RTI184.

FIG. 6 shows images showing the positive effects on growth and vigor in pepper as a result of addition of Bacillus licheniformis strain RTI184 to PROMIX BX (PREMIER TECH, INC; Quebec, Canada) potting soil limed to pH of 6.5 according to one or more embodiments of the present invention. A) Pepper plants after addition to soil of 1×10⁷ CFU/g Bacillus licheniformis RTI184 spores; and B) Control pepper plants in the same soil without addition of Bacillus licheniformis RTI184.

FIG. 7 is a schematic diagram showing both previously reported Fengycin-type and Dehydroxyfengycin-type cyclic lipopeptides produced by microbial species including Bacillus licheniformis and newly identified (shown in bold type) Fengycin- and Dehydroxyfengycin-type molecules produced by the Bacillus licheniformis RTI184 isolate according to one or more embodiments of the present invention.

FIG. 8 is an image of agarose gel electrophoresis of BOX-PCR fingerprinting patterns for genomic DNA of Bacillus licheniformis strains CH200, RTI1242, RTI1249, RTI184, RTI1243, RTI1112, FCC1598, RTI239, RTI241, and RTI253 according to one or more embodiments of the present invention. The 1 kb DNA ladder (FERMENTAS) was used as molecular size marker. Based on the BOX-PCR patterns, the ten strains fall into three main groups, Group 1, Group 2A-2B, (Group 2A and 2B represent the position on the gel) and Group 3, which comprises the strains not belonging to the Groups 1 and 2.

FIG. 9 shows images showing the positive growth effects of treatment of potato plants grown in Globodera-infected soil with spores of Bacillus licheniformis strain RTI184 according to one or more embodiments of the present invention. Potato plants after 48 days growth are shown in the figure. A) Control plants; and B) Plants treated with RTI184 spores.

### DETAILED DESCRIPTION OF THE INVENTION

The terms “a,” “an,” and “the” refer to “one or more” when used in this application, including the claims. Thus, for example, reference to “a plant” includes a plurality of plants, unless the context clearly is to the contrary.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

In certain embodiments of the present invention, compositions and methods are provided for benefiting plant growth and conferring protection against or controlling plant pathogenic infection. A plant-associated bacterium, identified as belonging to the species Bacillus licheniformis, was isolated from the root of rice grown in California and subsequently tested for plant growth promoting properties. More specifically, the isolated bacterial strain was identified as a new strain of Bacillus licheniformis through sequence analysis of highly conserved 16S rRNA and rpoB genes (see EXAMPLE 1). The 16S RNA sequence of the new bacterial isolate (designated “RTI184”) was determined to be nearly identical to the 16S rRNA gene sequence of two other known strains of B. licheniformis, Bacillus licheniformis strain 9945A (99%, 2 bp difference over 1545 bp in one copy of the 16S rRNA gene out of three different copies) and Bacillus licheniformis ATCC 14580 (99%, 8 bp difference over 1545 bp). In addition, it was determined that the rpoB sequence of RTI184 has 100% sequence identity to known strain Bacillus licheniformis 9945A (CP005965) and 97% sequence identity to Bacillus licheniformis strain deposited as ATCC 14580 (97 bp difference over 3015 bp). To further discriminate between strain RTI184 and Bacillus licheniformis 9945A, the genome sequences for their pathways involved in biosynthesis of lichenysin, the characteristic anionic cyclic lipoheptapeptide biosurfactant produced by Bacillus licheniformis species, were compared. Although similar, some differences were observed between the lichA and lichB genes for strains RTI184 and 9945A. Thus, the RTI184 strain was identified as a unique strain of Bacillus licheniformis. The strain of B. licheniformis RTI 184 was deposited on 13 Nov. 2014 under the terms of the Budapest Treaty on the International Recognition of the Deposit of Microorganisms for the Purposes of Patent Procedure at the American Type Culture Collection (ATCC) in Manassas, Va., USA and bears the Patent Accession No. PTA-121722.

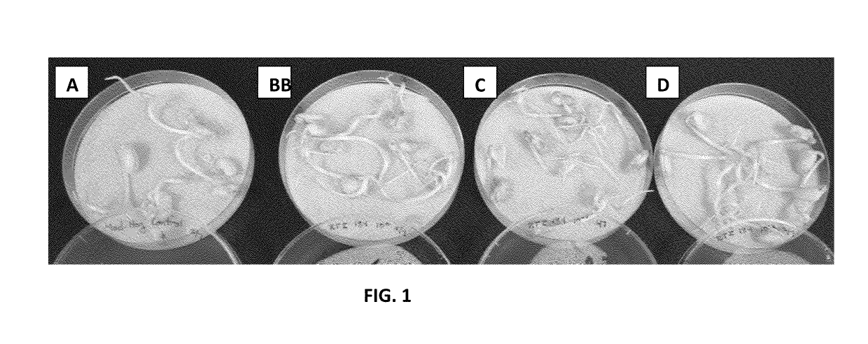

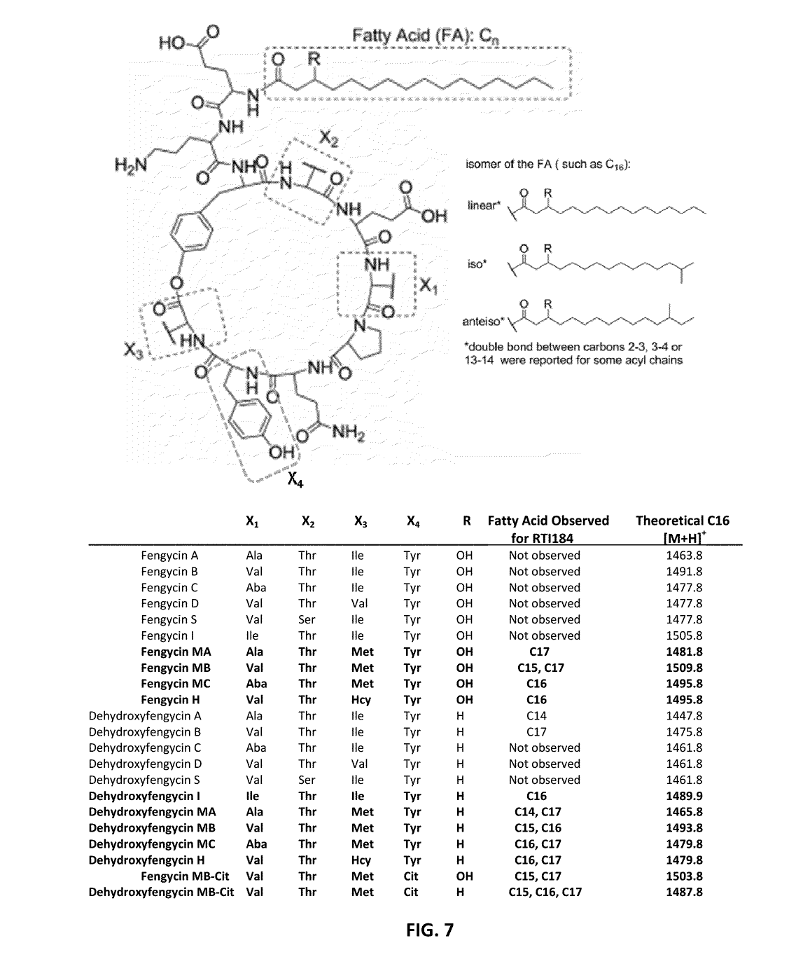

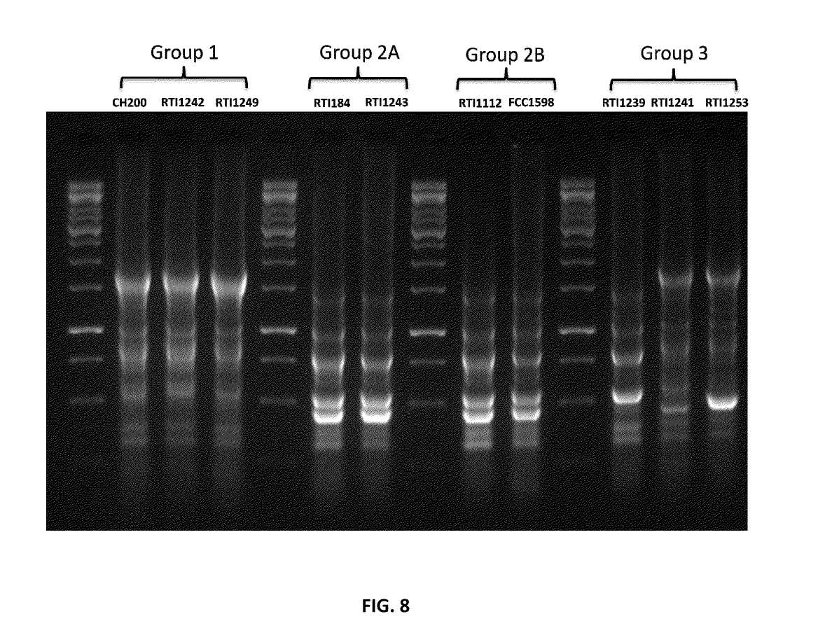

Experiments were performed that showed substantial growth promoting activity of the Bacillus licheniformis RTI184 strain in a wide range of plant species. In addition, experiments were performed to investigate the types of cyclic lipopeptides (i.e., cyclic peptide molecules that contain a fatty acid group known as Fengycins and Dehydroxyfengycins) that are produced by the Bacillus licheniformis strain RTI184. The experimental results for the RTI184 strain are provided in FIGS. 1-9 and in EXAMPLES 2-9 herein below. Surprisingly, the investigations of the cyclic lipopeptides resulted in the discovery that the RTI184 strain produces 4 classes of previously unreported Fengycin- and Dehydroxyfengycin-type molecules. The new classes are designated as: 1) Fengycin H and Dehydroxyfengycin H; 2) Dehydroxyfengycin I; 3) Fengycin MA/MB/MC and Dehydroxyfengycin MA/MB/MC; and 4) Fengycin MB-Cit and Dehydroxyfengycin MB-Cit, the details of which are described in EXAMPLE 7 and shown in FIG. 7. In addition to the discovery of the new classes of cyclic lipopeptides produced by the RTI184 strain, experiments revealed that synthesis of these new types of Fengycin- and Dehydroxyfengycin-type metabolites is strain dependent rather than intrinsic to the species Bacillus licheniformis. For example, even closely related Bacillus licheniformis strains produced different Fengycin- and Dehydroxyfengycin-type molecules and one closely related Bacillus licheniformis strain failed to produce any Fengycin- or Dehydroxyfengycin-type metabolites at all (see EXAMPLE 7 and FIG. 8). Thus, the newly discovered Bacillus licheniformis RTI184 strain possesses unique properties for benefiting plant growth and health not uniformly exhibited among Bacillus licheniformis strains.

The experimental results showing the antimicrobial properties of the Bacillus licheniformis RTI184 strain against common plant pathogenic organisms are described in EXAMPLE 2 and phenotypic traits such as phytohormone production, acetoin and indole acetic acid (IAA), and nutrient cycling of the strain are described in EXAMPLE 3.

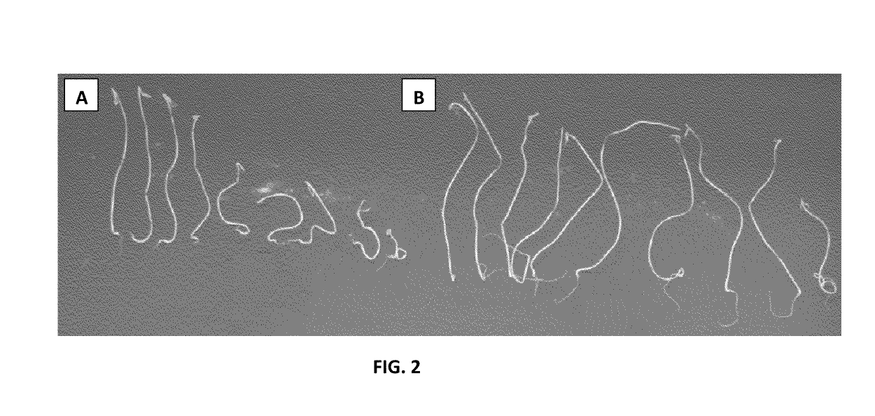

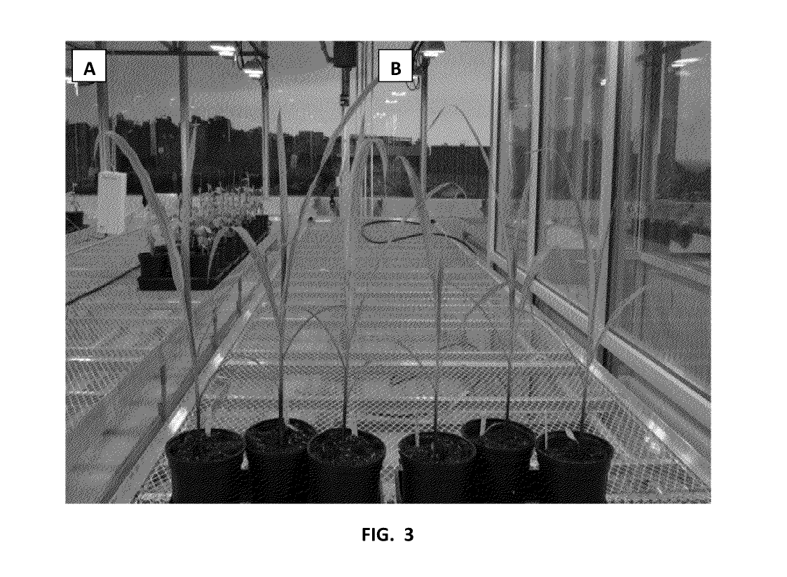

EXAMPLEs 4 and 5 describe the positive effects of inoculation of seed from a variety of plants with negative cells and spores of the Bacillus licheniformis RTI184 strain on seed germination, root development, and early growth. The results are shown in Tables III-IV and FIGS. 1-3. As an illustration, FIGS. 1A-1D are images of soybean seeds showing the positive effects on root hair development after inoculation by vegetative cells of RTI184 at a concentrations ranging from 10⁶ to 10⁸ after 7 days of growth as compared to untreated control. The data show that addition of the RTI184 cells stimulated formation of lateral roots and fine root hairs compared to uninoculated control seeds. Fine root hairs are important in the uptake of water, nutrients and plant interaction with other microorganisms in the rhizosphere. FIG. 2 shows similar positive effects on root development after of inoculation of MONEY MAKER tomato seed with spores of the RTI184 strain. FIG. 3 shows the early growth promoting activity of the RTI184 isolate in corn. Germinated corn seeds were inoculated for 2 days in a suspension of ^{˜}2×10⁷ CFU/ml of the RTI184 strain and subsequently planted in pots. The beneficial effects of the RTI184 strain on early growth in corn are shown in the images in FIG. 3. FIG. 3A shows 8 week-old plants inoculated with RTI184 and FIG. 3B shows control plants. Dry weight of the corn seedlings was determined after 8 weeks of growth resulting in a 25% increase in dry weight over the non-inoculated control for the RTI184 treated plants.

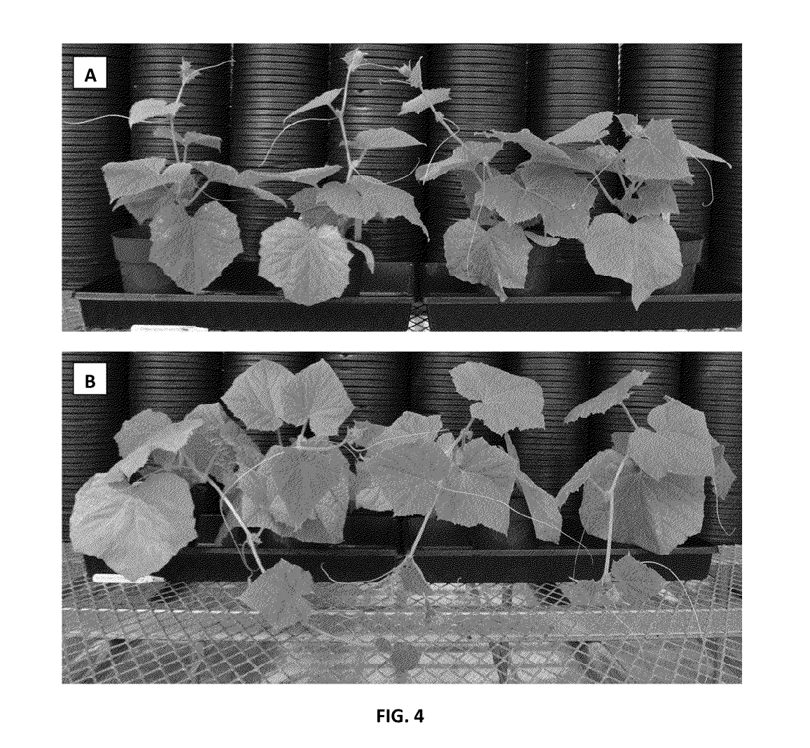

The effect on growth and vigor in cucumber, tomato, and pepper upon addition of the bacterial isolate RTI184 to soil is described in EXAMPLE 6. In this experiment, cucumber, tomato, and pepper seeds were planted in PROMIX BX (PREMIER TECH, INC; Quebec, Canada) potting soil, limed to a pH of 6.5, and enhanced with 1×10⁷ spores/g Bacillus licheniformis strain RTI184. Plants were imaged and harvested and their dry shoot weight was measured and compared to data obtained for non-inoculated control plants. RTI184 outperformed the control for all crop types. The positive effects of the RTI184 strain on growth are shown in FIGS. 4-6 and in Table V. The increase in dry shoot mass observed for cucumber, tomato, and pepper for RTI184-enhanced soil over control was 44%, 68%, and 26%, respectively.

EXAMPLE 7 describes the investigation of the cyclic lipopeptides, Fengycins and Dehydroxyfengycins, produced by the Bacillus licheniformis RTI184 strain, and surprisingly, the identification of 4 previously unreported classes of these molecules. FIG. 7 is a schematic diagram showing both previously reported Fengycin-type and Dehydroxyfengycin-type cyclic lipopeptides produced by microbial species including Bacillus licheniformis and newly identified Fengycin- and Dehydroxyfengycin-type molecules produced by the Bacillus licheniformis RTI184 isolate (shown in bold type). A summary of the previously reported Fengycin- and Dehydroxyfengycin-type lipopeptides and the newly identified metabolites produced by the RTI184 strain is provided in Tables VI and VII. For the first new class, it was determined that the RTI184 strain produces previously unidentified derivatives of these compounds where the L-isoleucine at position 8 of the cyclic peptide chain (referred to as X₃ in FIG. 7) is replaced by L-methionine. This new class of Fengycin and Dehydroxyfengycin are referred to herein as MA, MB and MC, referring to derivatives of classes A, B and C in which the L-isoleucine at X₃ in FIG. 7 has been replaced by L-methionine. The newly identified molecules are shown in bold in FIG. 7 and in Table VI. In addition, another previously unidentified class of these molecules produced by the Bacillus licheniformis strain RTI184 was identified, in which the Tyrosine (Tyr) of Fengycin MB and Dehydroxyfengycin MB (position X₄ in FIG. 7) is replaced by the α-amino acid, Citruline. This new class of Fengycin and Dehydroxyfengycin is being referred to herein as Fengycin MB-Cit and Dehydroxyfengycin MB-Cit and is shown in bold in FIG. 7 and in Table VI. It was further determined that the Bacillus licheniformis strain RTI184 produces an additional class of Fengycin and Dehydroxyfengycin that has not been previously identified. In this class, the L-isoleucine of Fengycin B and Dehydroxyfengycin B (position X₃ in FIG. 7) is replaced by L-homo-cysteine (Hcy). These previously unidentified Fengycin and Dehydroxyfengycin metabolites are referred to herein as Fengycin H and Dehydroxyfengycin H and are shown in bold in FIG. 7 and in Table VI. It was further determined that the Bacillus licheniformis strain RTI184 produces an additional class of Dehydroxyfengycin that has not been previously identified. In this class, position X₁ in FIG. 7 is replaced by L-isoleucine. This previously unidentified Dehydroxyfengycin metabolite is referred to herein as Dehydroxyfengycin I and is shown in bold in FIG. 7 and in Table VI.

In addition to the discovery of the new classes of cyclic lipopeptides produced by the RTI184 strain, further experiments described in EXAMPLE 7 revealed that synthesis of these new types of Fengycin- and Dehydroxyfengycin-type metabolites is strain dependent rather than intrinsic to the species Bacillus licheniformis. For these experiments, the synthesis of cyclic lipopeptides was compared between ten Bacillus licheniformis strains. The ten bacterial strains selected for this analysis were identified as being Bacillus licheniformis strains based on sequence comparison of their highly conserved 16S rRNA and rpoB gene sequences. The genomic DNA of each strain was isolated and compared by BOX-PCR pattern and an image of the gel showing the resulting BOX-PCR patterns for the strains is shown in FIG. 8. Specifically, FIG. 8 shows agarose gel electrophoresis of BOX-PCR fingerprinting patterns for genomic DNA of Bacillus licheniformis strains CH200, RTI1242, RTI1249, RTI184, RTI1243, RTI1112, FCC1598, and RTI239, RTI241, and RTI253. Based on their BOX-PCR pattern, the ten strains fell into three main groups, Group 1, Group 2A-2B (Group 2A and 2B represent the position on the gel in FIG. 8), and Group 3, which comprises the strains not belonging to the Groups 1 and 2.

To determine the type of Fengycin- and Dehydroxyfengycin-type metabolites produced by each of the ten Bacillus licheniformis strains, the strains were analyzed using UHPLC-TOF MS. In addition, the Lichenysin-type metabolites, characteristic for Bacillus licheniformis, were also analyzed as internal controls. The results of the UHPLC-TOF MS analysis are summarized in Table VII in EXAMPLE 7. The lichenysin and fengycin-type and dehyroxyfengycin-type molecules, their lipid modification (fatty acid (FA) chain length), predicted molecular mass, and their presence or absence in the culture supernatant of each of the ten Bacillus licheniformis strains are presented in Table VII.

The data show that the Lichenysin-type metabolites were synthesized by all ten strains, confirming that they are Bacillus licheniformis strains. On the other hand, major differences were observed between the ten strains with regard to the production of the Fengycin- and Dehydroxyfengycin-type metabolites. Strains RTI184 and RTI1112 (Group 2), which had identical BOX-PCR patterns, were found to produce the same type of Fengycin- and Dehydroxyfengycin-type metabolites, including dehydroxy Fengycin A/B/C/D/I/S, Dehydroxyfengycin H/MA/MB/MC, dehydroxyfengycin MB-Cit, Fengycin H/MA/MB/MC and Fengycin MB-Cit, but failed to produce the Fengycin A/B/C/D/I/S type metabolites. On the other hand, strain FCC1598 which also falls into Group 2, produced the Fengycin A/B/C/D/I/S type metabolites, but failed to produce the Fengycin H/MA/MB/MC-type metabolites. Surprisingly, strain RTI1243, which also belongs to Group 2, did not produce any of the Fengycin- and Dehydroxyfengycin-type metabolites. Finally, two of the strains belonging to Group 1 (RTI1242 and RTI1249) and two strains belonging to Group 3 (RTI1241 and RTI1253) failed to produce any of the Fengycin- and Dehydroxyfengycin-type metabolites, whereas the CH 200 and RTI1239, belonging to Group 1 and Group 3, respectively, produced all of the Fengycin- and Dehydroxyfengycin-type metabolites. Based on these results, it was concluded that the synthesis of the different types of Fengycin- and Dehydroxyfengycin-type metabolites, including the newly identified citruline-containing metabolites, is strain dependent rather than intrinsic to the species Bacillus licheniformis. For example, even closely related Bacillus licheniformis Group 2 strains produced different Fengycin- and Dehydroxyfengycin-type molecules and one closely related Group 2 strain failed to produce any Fengycin- or Dehydroxyfengycin-type metabolites at all.

The positive effect on yield in squash, broccoli, turnip, and strawberry upon addition of RTI184 spores to soil by drip irrigation is described in EXAMPLE 8. In these field trial experiments, drip irrigation was used to apply 1.5×10¹¹, 2.5×10¹², or 2.5×10¹³ CFU/hectare of B. licheniformis RTI184 spores at the time of planting, and again 2 weeks later. As compared to control squash plants in which B. licheniformis RTI184 spores were not included in the irrigation, addition of the RTI184 spores at all concentrations resulted in an increase in yield for both total and marketable squash. Specifically, RTI184 treated plants (application rate 2.5×10¹² CFU/hectare) resulted in an average of 33 kg of total squash of which 26 kg was marketable, as compared to 22 kg of total squash of which 17 kg was marketable for the untreated control plants. This is a 50% increase in weight of total squash and a 53% increase in weight of marketable squash. The substantial increase in both total squash weight and marketable squash weight of the plants treated with RTI184 relative to the control plants demonstrates the positive growth effect provided by treatment with the RTI184 spores.

As compared to control broccoli plants in which B. licheniformis RTI184 spores were not included in the irrigation, addition of the RTI184 spores resulted in a consistent increase in broccoli fresh weight yield from 3 kg (control) to 4 kg (2.5×10¹³ CFU/hectare RTI184), 3.9 kg (2.5×10¹² CFU/hectare RTI184), and 4.6 kg (1.5×10¹¹ CFU/hectare RTI184) or a 33%, 30%, and 53% increase in weight, respectively. The substantial increase in fresh weight of the plants treated with RTI184 relative to the control plants demonstrates the positive growth effect provided by treatment with the RTI184 spores.

As compared to control turnip plants in which B. licheniformis RTI184 spores were not included in the irrigation, addition of the RTI184 spores at all concentrations resulted in a consistent increase in turnip tuber weight yield from 3 kgs (control) to approximately 5.3 kgs which is a 60% increase. The substantial increase in tuber weight of the plants treated with RTI184 relative to the control plants demonstrates the positive growth effect provided by treatment with the RTI184 spores.

As compared to control strawberry plants in which B. licheniformis RTI184 spores were not included in the irrigation, addition of the RTI184 spores resulted in a increase in total strawberry yield of 5% (1.5×10¹¹ CFU/hectare RTI184), 8% (2.5×10¹² CFU/hectare RTI184), and 11% (2.5×10¹³ CFU/hectare RTI184). The increase in yield of the plants treated with RTI184 relative to the control plants demonstrates the positive growth effect provided by treatment with the RTI184 spores.

A similar field trial was performed in which lettuce plants were drip irrigated with 12.5×10¹² CFU/hectare of B. licheniformis RTI184 spores at the time of planting and again 2 weeks later. As compared to control plants in which B. licheniformis RTI184 spores were not included in the irrigation, addition of the RTI184 spores resulted in a consistent increase in lettuce weight yield from 45.6 kgs (control) to 52.8 kgs, which is a 16% increase. The increased weight of the plants treated with RTI184 relative to the control plants demonstrates the positive growth effect provided by treatment with the RTI184 spores.

EXAMPLE 9 describes the positive growth effect provided by treatment of potato plants grown in nematode-infected soil with RTI184 spores. Images showing the increased size of the plants treated with RTI184 spores as compared to untreated control plants are shown in FIG. 8B and FIG. 8A, respectively. The increased size of the plants treated with RTI184 relative to the control plants demonstrates the positive growth effect provided by treatment with the RTI184 spores.

In embodiments of the present invention, compositions and methods are provided that include a new strain of Bacillus licheniformis having plant growth promoting activity and designated RTI184 having ATCC Accession No. PTA-121722. The compositions and methods of the presently disclosed subject matter are useful for benefiting plant growth when applied to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium. The compositions containing the Bacillus licheniformis RTI184 strain of the present invention are useful for lowering the need for nitrogen containing fertilizers and soluble minerals, increasing the availability of plant nutrients, and competing against plant pathogens, thus increasing overall plant health and decreasing the need for chemical fungicides and pesticides. The compositions containing the Bacillus licheniformis RTI184 strain can be used in combination with one or more chemical agents including, for example, insecticides, herbicides, fungicides, nematicides, bacteriocides, plant growth regulators, and fertilizers.

Beneficial plant associated bacteria, both rhizospheric and endophytic, are known to provide a multitude of benefits to host plants that ranges from resistance to diseases and insects pests and tolerance to environmental stresses including cold, salinity and drought stress. As the plants with inoculated plant growth promoting bacteria aquire more water and nutrient from soils, e.g. due to a better developed root system, the plants grow healthier and are less susceptible to biotic and abiotic stresses. As such the microbial compositions of the present invention can be applied alone or in combination with current crop management inputs such as chemical fertilizers, herbicides, and pesticides to maximize crop productivity. Plant growth promoting effects translate into faster growing plants and increase above ground biomass, a property that can be applied to improve early vigor. One benefit of improved early vigor is that plants are more competitive and out-compete weeds, which directly reduces the cost for weed management by minimizing labor and herbicide-application. Plant growth promoting effects also translate into improved root development, including deeper and wider roots with more fine roots that are involved in the uptake of water and nutrients. This property allows for better use of agricultural resources, and a reduction in water used in irrigation needs and/or fertilizer application. Changes in root development and root architecture affect the interactions of the plant with other soil-borne microorganisms, including beneficial fungi and bacteria that help the plant with nutrient uptake including nitrogen fixation and phosphate solubilization. These beneficial microbes also compete against plant pathogens to increase overall plant health and decrease the need for chemical fungicides and pesticides.

In one embodiment of the present invention, a composition for benefiting plant growth is provided including a biologically pure culture of Bacillus licheniformis RTI184 deposited as ATCC No. PTA-121722, or a mutant thereof having all the identifying characteristics thereof, in an amount suitable to benefit plant growth. The Bacillus licheniformis RTI184 can be in the form of spores or in the form of vegetative cells. The composition benefits plant growth when applied to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

The phrase “a biologically pure culture of a Bacillus licheniformis RTI184” refers to one or a combination of: spores of the biologically pure fermentation culture of a bacterial strain, vegetative cells of the biologically pure fermentation culture of a bacterial strain, one or more products of the biologically pure fermentation culture of a bacterial strain, a culture solid of the biologically pure fermentation culture of a bacterial strain, a culture supernatant of the biologically pure fermentation culture of a bacterial strain, an extract of the biologically pure fermentation culture of the bacterial strain, and one or more metabolites of the biologically pure fermentation culture of a bacterial strain.

The growth benefit of the plant can be exhibited by one or a combination of improved seedling vigor, improved root development, improved plant growth, improved plant health, increased yield, or improved appearance.

The compositions and methods of the present invention are beneficial to a wide range of plants including, but not limited to, monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

In one or more embodiments, the plant can include soybean, wheat, cotton, corn, tomato, squash, cucumber, grass, turf grass, ornamental plants, hydrangea, or poinsettia.

The composition can be in the form of a liquid, an oil dispersion, a dust, a dry wettable powder, a spreadable granule, or a dry wettable granule. The composition can be in the form of a liquid or an oil dispersion and the Bacillus licheniformis RTI184 can be present at a concentration of from about 1.0×10⁹ CFU/ml to about 1.0×10¹² CFU/ml. The composition can be in the form of a dust, a dry wettable powder, a spreadable granule, or a dry wettable granule and the Bacillus licheniformis RTI184 can present in an amount of from about 1.0×10⁹ CFU/g to about 1.0×10¹² CFU/g. The composition can be in the form of an oil dispersion and the Bacillus licheniformis RTI184 can be present at a concentration of from about 1.0×10⁹ CFU/ml to about 1.0×10¹² CFU/ml. The amount of the Bacillus licheniformis RTI184 suitable to benefit plant growth can range from about 1.0×10⁸ CFU/ha to about 1.0×10¹³ CFU/ha.

The composition for benefiting plant growth including a biologically pure culture of the Bacillus licheniformis RTI184 can be in a form of a planting matrix. The planting matrix can be in the form of a potting soil.

The composition can further include one or a combination of a microbial or a chemical insecticide, fungicide, nematicide, bacteriocide, herbicide, plant extract, plant growth regulator, or fertilizer present in an amount suitable to benefit plant growth and/or to confer protection against a pathogenic infection in a susceptible plant. The insecticide can include bifenthrin. The nematicide can include cadusafos. The insecticide can include bifenthrin and clothianidin. The composition can be formulated as a liquid and the insecticide can include bifenthrin or zeta-cypermethrin.

In one embodiment of the present invention, a coated plant seed is provided, the plant seed coated with a composition comprising spores of the biologically pure culture of Bacillus licheniformis RTI184 deposited as ATCC No. PTA-121722, or a mutant thereof having all the identifying characteristics thereof, present in an amount suitable to benefit plant growth. The coated plant seed can include an amount of Bacillus licheniformis spores ranging from about 1.0×10² CFU/seed to about 1.0×10⁹ CFU/seed.

The plant seed can include, but is not limited to, seed of a the seed of monocots, dicots, Cereals, Corn, Sweet Corn, Popcorn, Seed Corn, Silage Corn, Field Corn, Rice, Wheat, Barley, Sorghum, Brassica Vegetables, Broccoli, Cabbage, Cauliflower, Brussels Sprouts, Collards, Kale, Mustard Greens, Kohlrabi, Bulb Vegetables, Onion, Garlic, Shallots, Fruiting Vegetables, Pepper, Tomato, Eggplant, Ground Cherry, Tomatillo, Okra, Grape, Herbs/Spices, Cucurbit Vegetables, Cucumber, Cantaloupe, Melon, Muskmelon, Squash, Watermelon, Pumpkin, Eggplant, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Cotton, Flax, Peanut, Rapeseed, Safflower, Sesame, Sunflower, Soybean, Root/Tuber and Corm Vegetables, Carrot, Potato, Sweet Potato, Beets, Ginger, Horseradish, Radish, Ginseng, Turnip, sugarcane, sugarbeet, Grass, or Turf grass.

The coated seed can further include one or a combination of an insecticide, a fungicide, a nematicide, a bacteriocide, a plant growth regulator, or a fertilizer present in an amount suitable to benefit plant growth. The insecticide can include bifenthrin. The nematicide can include cadusafos. The insecticide can include bifenthrin and clothianidin.

In one embodiment of the present invention, a composition is provided for benefiting plant growth, the composition including the biologically pure culture of Bacillus licheniformis RTI184 deposited as ATCC No. PTA-121722, or mutant thereof having all the identifying characteristics thereof; and one or more chemical active agent including an insecticide, a herbicide, a fungicide, a nematicide, a bacteriocide, a plant growth regulator, or a fertilizer.

The composition can be in the form of a liquid, an oil dispersion, a dry wettable powder, a spreadable granule, or a dry wettable granule. The Bacillus licheniformis RTI184 can be in the form of spores or in the form of vegetative cells. The composition can be in the form of a liquid or an oil dispersion and the Bacillus licheniformis RTI184 can be present at a concentration of from about 1.0×10⁹ CFU/ml to about 1.0×10¹² CFU/ml. The composition can be in the form of a dust, a dry wettable powder, a spreadable granule, or a dry wettable granule and the Bacillus licheniformis RTI184 can be present in an amount of from about 1.0×10⁹ CFU/g to about 1.0×10¹² CFU/g.

The insecticide can include bifenthrin. The nematicide can include cadusafos. The insecticide can include bifenthrin and clothianidin. The composition can be formulated as a liquid and the insecticide can include bifenthrin or zeta-cypermethrin.

The insecticide can be bifenthrin and the composition formulation can further comprise a hydrated aluminum-magnesium silicate, and at least one dispersant selected from the group consisting of a sucrose ester, a lignosulfonate, an alkylpolyglycoside, a naphthalenesulfonic acid formaldehyde condensate and a phosphate ester. The bifenthrin insecticide can be present at a concentration ranging from 0.1 g/ml to 0.2 g/ml. The bifenthrin insecticide can be present at a concentration of about 0.1715 g/ml. The rate of application of the bifenthrin insecticide can be in the range of from about 0.1 gram of bifenthrin per hectare (g ai/ha) to about 1000 g ai/ha, more preferably in a range of from about 1 g ai/ha to about 100 g ai/ha.

In addition, in one or more embodiments, suitable insecticides, herbicides, fungicides, and nematicides of the compositions and methods of the present invention can include the following:

Insecticides: A0) various insecticides, including agrigata, al-phosphide, amblyseius, aphelinus, aphidius, aphidoletes, artimisinin, autographa californica NPV, azocyclotin Bacillus subtilis, Bacillus thuringiensis-spp. aizawai, Bacillus thuringiensis spp. kurstaki, Bacillus thuringiensis, Beauveria, Beauveria bassiana, betacyfluthrin, biologicals, bisultap, brofluthrinate, bromophos-e, bromopropylate, Bt-Corn-GM, Bt-Soya-GM, capsaicin, cartap, celastrus-extract, chlorantraniliprole, chlorbenzuron, chlorethoxyfos, chlorfluazuron, chlorpyrifos-e, cnidiadin, cryolite, cyanophos, cyantraniliprole, cyhalothrin, cyhexatin, cypermethrin, dacnusa, DCIP, dichloropropene, dicofol, diglyphus, diglyphus+dacnusa, dimethacarb, dithioether, dodecyl-acetate, emamectin, encarsia, EPN, eretmocerus, ethylene-dibromide, eucalyptol, fatty-acids, fatty-acids/salts, fenazaquin, fenobucarb (BPMC), fenpyroximate, flubrocythrinate, flufenzine, formetanate, formothion, furathiocarb, gamma-cyhalothrin, garlic-juice, granulosis-virus, harmonia, heliothis armigera NPV, inactive bacterium, indol-3-ylbutyric acid, iodomethane, iron, isocarbofos, isofenphos, isofenphos-m, isoprocarb, isothioate, kaolin, lindane, liuyangmycin, matrine, mephosfolan, metaldehyde, metarhizium-anisopliae, methamidophos, metolcarb (MTMC), mineral-oil, mirex, m-isothiocyanate, monosultap, myrothecium verrucaria, naled, neochrysocharis formosa, nicotine, nicotinoids, oil, oleic-acid, omethoate, orius, oxymatrine, paecilomyces, paraffin-oil, parathion-e, pasteuria, petroleum-oil, pheromones, phosphorus-acid, photorhabdus, phoxim, phytoseiulus, pirimiphos-e, plant-oil, plutella xylostella GV, polyhedrosis-virus, polyphenol-extracts, potassium-oleate, profenofos, prosuler, prothiofos, pyraclofos, pyrethrins, pyridaphenthion, pyrimidifen, pyriproxifen, quillay-extract, quinomethionate, rape-oil, rotenone, saponin, saponozit, sodium-compounds, sodium-fluosilicate, starch, steinernema, streptomyces, sulfluramid, sulphur, tebupirimfos, tefluthrin, temephos, tetradifon, thiofanox, thiometon, transgenics (e.g., Cry3Bb1), triazamate, trichoderma, trichogramma, triflumuron, verticillium, vertrine, isomeric insecticides (e.g., kappa-bifenthrin, kappa-tefluthrin), dichoromezotiaz, broflanilide, pyraziflumid; A1) the class of carbamates, including aldicarb, alanycarb, benfuracarb, carbaryl, carbofuran, carbosulfan, methiocarb, methomyl, oxamyl, pirimicarb, propoxur and thiodicarb; A2) the class of organophosphates, including acephate, azinphos-ethyl, azinphos-methyl, chlorfenvinphos, chlorpyrifos, chlorpyrifos-methyl, demeton-S-methyl, diazinon, dichlorvos/DDVP, dicrotophos, dimethoate, disulfoton, ethion, fenitrothion, fenthion, isoxathion, malathion, methamidaphos, methidathion, mevinphos, monocrotophos, oxymethoate, oxydemeton-methyl, parathion, parathion-methyl, phenthoate, phorate, phosalone, phosmet, phosphamidon, pirimiphos-methyl, quinalphos, terbufos, tetrachlorvinphos, triazophos and trichlorfon; A3) the class of cyclodiene organochlorine compounds such as endosulfan; A4) the class of fiproles, including ethiprole, fipronil, pyrafluprole and pyriprole; A5) the class of neonicotinoids, including acetamiprid, clothianidin, dinotefuran, imidacloprid, nitenpyram, thiacloprid and thiamethoxam; A6) the class of spinosyns such as spinosad and spinetoram; A7) chloride channel activators from the class of mectins, including abamectin, emamectin benzoate, ivermectin, lepimectin and milbemectin; A8) juvenile hormone mimics such as hydroprene, kinoprene, methoprene, fenoxycarb and pyriproxyfen; A9) selective homopteran feeding blockers such as pymetrozine, flonicamid and pyrifluquinazon; A10) mite growth inhibitors such as clofentezine, hexythiazox and etoxazole; A11) inhibitors of mitochondrial ATP synthase such as diafenthiuron, fenbutatin oxide and propargite; uncouplers of oxidative phosphorylation such as chlorfenapyr; A12) nicotinic acetylcholine receptor channel blockers such as bensultap, cartap hydrochloride, thiocyclam and thiosultap sodium; A13) inhibitors of the chitin biosynthesis type 0 from the benzoylurea class, including bistrifluron, diflubenzuron, flufenoxuron, hexaflumuron, lufenuron, novaluron and teflubenzuron; A14) inhibitors of the chitin biosynthesis type 1 such as buprofezin; A15) moulting disruptors such as cyromazine; A16) ecdyson receptor agonists such as methoxyfenozide, tebufenozide, halofenozide and chromafenozide; A17) octopamin receptor agonists such as amitraz; A18) mitochondrial complex electron transport inhibitors pyridaben, tebufenpyrad, tolfenpyrad, flufenerim, cyenopyrafen, cyflumetofen, hydramethylnon, acequinocyl or fluacrypyrim; A19) voltage-dependent sodium channel blockers such as indoxacarb and metaflumizone; A20) inhibitors of the lipid synthesis such as spirodiclofen, spiromesifen and spirotetramat; A21) ryanodine receptor-modulators from the class of diamides, including flubendiamide, the phthalamide compounds (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, chloranthraniliprole and cy-anthraniliprole; A22) compounds of unknown or uncertain mode of action such as azadirachtin, amidoflumet, bifenazate, fluensulfone, piperonyl butoxide, pyridalyl, sulfoxaflor; or A23) sodium channel modulators from the class of pyrethroids, including acrinathrin, allethrin, bifenthrin, cyfluthrin, lambda-cyhalothrin, cypermethrin, alpha-cypermethrin, beta-cypermethrin, zeta-cypermethrin, deltamethrin, esfenvalerate, etofenprox, fenpropathrin, fenvalerate, flucythrinate, tau-fluvalinate, permethrin, silafluofen and tralomethrin.

Fungicides: B0) benzovindiflupyr, anitiperonosporic, ametoctradin, amisulbrom, copper salts (e.g., copper hydroxide, copper oxychloride, copper sulfate, copper persulfate), boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefaciens (e.g., strains QST 713, FZB24, MB1600, D747), extract from Melaleuca alternifolia, pyrisoxazole, oxpoconazole, etaconazole, fenpyrazamine, naftifine, terbinafine, validamycin, pyrimorph, valifenalate, fthalide, probenazole, isotianil, laminarin, estract from Reynoutria sachalinensis, phosphorous acid and salts, teclofthalam, triazoxide, pyriofenone, organic oils, potassium bicarbonate, chlorothalonil, fluoroimide; B1) azoles, including bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; B2) strobilurins, including azoxystrobin, dimoxystrobin, enestroburin, fluoxastrobin, kresoxim-methyl, methominostrobin, orysastrobin, picoxystrobin, pyraclostrobin, trifloxystrobin, enestroburin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; B3) carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl̂-methylthiazole-6-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3\4′-dichloro-4-fluorobiphenyl-2-yl)-3-difluoro-methyl-1-methylpyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methylthiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethylpyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1 H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-I H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1 H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-I H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1 H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; B4) heterocyclic compounds, including fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloro pyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; B5) carbamates, including mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; or B6) other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

Herbicides: C1) acetyl-CoA carboxylase inhibitors (ACC), for example cyclohexenone oxime ethers, such as alloxydim, clethodim, cloproxydim, cycloxydim, sethoxydim, tralkoxydim, butroxydim, clefoxydim or tepraloxydim; phenoxyphenoxypropionic esters, such as clodinafop-propargyl, cyhalofop-butyl, diclofop-methyl, fenoxaprop-ethyl, fenoxaprop-P-ethyl, fenthiapropethyl, fluazifop-butyl, fluazifop-P-butyl, haloxyfop-ethoxyethyl, haloxyfop-methyl, haloxyfop-P-methyl, isoxapyrifop, propaquizafop, quizalofop-ethyl, quizalofop-P-ethyl or quizalofop-tefuryl; or arylaminopropionic acids, such as flamprop-methyl or flamprop-isopropyl; C2 acetolactate synthase inhibitors (ALS), for example imidazolinones, such as imazapyr, imazaquin, imazamethabenz-methyl (imazame), imazamox, imazapic or imazethapyr; pyrimidyl ethers, such as pyrithiobac-acid, pyrithiobac-sodium, bispyribac-sodium. KIH-6127 or pyribenzoxym; sulfonamides, such as florasulam, flumetsulam or metosulam; or sulfonylureas, such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, halosulfuron-methyl, imazosulfuron, metsulfuron-methyl, nicosulfuron, primisulfuron-methyl, prosulfuron, pyrazosulfuron-ethyl, rimsulfuron, sulfometuron-methyl, thifensulfuron-methyl, triasulfuron, tribenuron-methyl, triflusulfuron-methyl, tritosulfuron, sulfosulfuron, foramsulfuron or iodosulfuron; C3) amides, for example allidochlor (CDAA), benzoylprop-ethyl, bromobutide, chiorthiamid. diphenamid, etobenzanidibenzchlomet), fluthiamide, fosamin or monalide; C4) auxin herbicides, for example pyridinecarboxylic acids, such as clopyralid or picloram; or 2,4-D or benazolin; C5) auxin transport inhibitors, for example naptalame or diflufenzopyr; C6) carotenoid biosynthesis inhibitors, for example benzofenap, clomazone (dimethazone), diflufenican, fluorochloridone, fluridone, pyrazolynate, pyrazoxyfen, isoxaflutole, isoxachlortole, mesotrione, sulcotrione (chlormesulone), ketospiradox, flurtamone, norflurazon or amitrol; C7) enolpyruvylshikimate-3-phosphate synthase inhibitors (EPSPS), for example glyphosate or sulfosate; C8) glutamine synthetase inhibitors, for example bilanafos (bialaphos) or glufosinate-ammonium; C9) lipid biosynthesis inhibitors, for example anilides, such as anilofos or mefenacet; chloroacetanilides, such as dimethenamid, S-dimethenamid, acetochlor, alachlor, butachlor, butenachlor, diethatyl-ethyl, dimethachlor, metazachlor, metolachlor, S-metolachlor, pretilachlor, propachlor, prynachlor, terbuchlor, thenylchlor or xylachlor; thioureas, such as butylate, cycloate, di-allate, dimepiperate, EPTC. esprocarb, molinate, pebulate, prosulfocarb, thiobencarb (benthiocarb), tri-allate or vemolate; or benfuresate or perfluidone; C10) mitosis inhibitors, for example carbamates, such as asulam, carbetamid, chlorpropham, orbencarb, pronamid (propyzamid), propham or tiocarbazil; dinitroanilines, such as benefin, butralin, dinitramin, ethalfluralin, fluchloralin, oryzalin, pendimethalin, prodiamine or trifluralin; pyridines, such as dithiopyr or thiazopyr; or butamifos, chlorthal-dimethyl (DCPA) or maleic hydrazide; C11) protoporphyrinogen IX oxidase inhibitors, for example diphenyl ethers, such as acifluorfen, acifluorfen-sodium, aclonifen, bifenox, chlomitrofen (CNP), ethoxyfen, fluorodifen, fluoroglycofen-ethyl, fomesafen, furyloxyfen, lactofen, nitrofen, nitrofluorfen or oxyfluorfen; oxadiazoles, such as oxadiargyl or oxadiazon; cyclic imides, such as azafenidin, butafenacil, carfentrazone-ethyl, cinidon-ethyl, flumiclorac-pentyl, flumioxazin, flumipropyn, flupropacil, fluthiacet-methyl, sulfentrazone or thidiazimin; or pyrazoles, such as ET-751.JV 485 or nipyraclofen; C12) photosynthesis inhibitors, for example propanil, pyridate or pyridafol; benzothiadiazinones, such as bentazone; dinitrophenols, for example bromofenoxim, dinoseb, dinoseb-acetate, dinoterb or DNOC; dipyridylenes, such as cyperquat-chloride, difenzoquat-methylsulfate, diquat or paraquat-dichloride; ureas, such as chlorbromuron, chlorotoluron, difenoxuron, dimefuron, diuron, ethidimuron, fenuron, fluometuron, isoproturon, isouron, linuron, methabenzthiazuron, methazole, metobenzuron, metoxuron, monolinuron, neburon, siduron or tebuthiuron; phenols, such as bromoxynil or ioxynil; chloridazon; triazines, such as ametryn, atrazine, cyanazine, desmein, dimethamethryn, hexazinone, prometon, prometryn, propazine, simazine, simetryn, terbumeton, terbutryn, terbutylazine or trietazine; triazinones, such as metamitron or metribuzin; uracils, such as bromacil, lenacil or terbacil; or biscarbamates, such as desmedipham or phenmedipham; C13) synergists, for example oxiranes, such as tridiphane; C14) CIS cell wall synthesis inhibitors, for example isoxaben or dichlobenil; C16) various other herbicides, for example dichloropropionic acids, such as dalapon; dihydrobenzofurans, such as ethofumesate; phenylacetic acids, such as chlorfenac (fenac); or aziprotryn, barban, bensulide, benzthiazuron, benzofluor, buminafos, buthidazole, buturon, cafenstrole, chlorbufam, chlorfenprop-methyl, chloroxuron, cinmethylin, cumyluron, cycluron, cyprazine, cyprazole, dibenzyluron, dipropetryn, dymron, eglinazin-ethyl, endothall, ethiozin, flucabazone, fluorbentranil, flupoxam, isocarbamid, isopropalin, karbutilate, mefluidide, monuron, napropamide, napropanilide, nitralin, oxaciclomefone, phenisopham, piperophos, procyazine, profluralin, pyributicarb, secbumeton, sulfallate (CDEC), terbucarb, triaziflam, triazofenamid or trimeturon; or their environmentally compatible salts.

Nematicides or bionematicides: Benomyl, cloethocarb, aldoxycarb, tirpate, diamidafos, fenamiphos, cadusafos, dichlofenthion, ethoprophos, fensulfothion, fosthiazate, heterophos, isamidofof, isazofos, phosphocarb, thionazin, imicyafos, mecarphon, acetoprole, benclothiaz, chloropicrin, dazomet, fluensulfone, 1,3-dichloropropene (telone), dimethyl disulfide, metam sodium, metam potassium, metam salt (all MITC generators), methyl bromide, biological soil amendments (e.g., mustard seeds, mustard seed extracts), steam fumigation of soil, allyl isothiocyanate (AITC), dimethyl sulfate, furfual (aldehyde).

Suitable plant growth regulators of the present invention include the following: Plant Growth Regulators: D1) Antiauxins, such as clofibric acid, 2,3,5-tri-iodobenzoic acid; D2) Auxins such as 4-CPA, 2,4-D, 2,4-DB, 2,4-DEP, dichlorprop, fenoprop, IAA, IBA, naphthaleneacetamide, α-naphthaleneacetic acids, 1-naphthol, naphthoxyacetic acids, potassium naphthenate, sodium naphthenate, 2,4,5-T; D3) cytokinins, such as 2iP, benzyladenine, 4-hydroxyphenethyl alcohol, kinetin, zeatin; D4) defoliants, such as calcium cyanamide, dimethipin, endothal, ethephon, merphos, metoxuron, pentachlorophenol, thidiazuron, tribufos; D5) ethylene inhibitors, such as aviglycine, 1-methylcyclopropene; D6) ethylene releasers, such as ACC, etacelasil, ethephon, glyoxime; D7) gametocides, such as fenridazon, maleic hydrazide; D8) gibberellins, such as gibberellins, gibberellic acid; D9) growth inhibitors, such as abscisic acid, ancymidol, butralin, carbaryl, chlorphonium, chlorpropham, dikegulac, flumetralin, fluoridamid, fosamine, glyphosine, isopyrimol, jasmonic acid, maleic hydrazide, mepiquat, piproctanyl, prohydrojasmon, propham, tiaojiean, 2,3,5-tri-iodobenzoic acid; D10) morphactins, such as chlorfluren, chlorflurenol, dichlorflurenol, flurenol; D11) growth retardants, such as chlormequat, daminozide, flurprimidol, mefluidide, paclobutrazol, tetcyclacis, uniconazole; D12) growth stimulators, such as brassinolide, brassinolide-ethyl, DCPTA, forchlorfenuron, hymexazol, prosuler, triacontanol; D13) unclassified plant growth regulators, such as bachmedesh, benzofluor, buminafos, carvone, choline chloride, ciobutide, clofencet, cyanamide, cyclanilide, cycloheximide, cyprosulfamide, epocholeone, ethychlozate, ethylene, fuphenthiourea, furalane, heptopargil, holosulf, inabenfide, karetazan, lead arsenate, methasulfocarb, prohexadione, pydanon, sintofen, triapenthenol, trinexapac.

The fertilizer can be a liquid fertilizer. The term “liquid fertilizer” refers to a fertilizer in a fluid or liquid form containing various ratios of nitrogen, phosphorous and potassium (for example, but not limited to, 10% nitrogen, 34% phosphorous and 0% potassium) and micronutrients, commonly known as starter fertilizers that are high in phosphorus and promote rapid and vigorous root growth.

Chemical formulations of the present invention can be in any appropriate conventional form, for example an emulsion concentrate (EC), a suspension concentrate (SC), a suspo-emulsion (SE), a capsule suspension (CS), a water dispersible granule (WG), an emulsifiable granule (EG), a water in oil emulsion (EO), an oil in water emulsion (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a dispersible concentrate (DC), a wettable powder (WP) or any technically feasible formulation in combination with agriculturally acceptable adjuvants.

In one embodiment of the present invention, a method is provided for benefiting plant growth, the method including delivering a composition including the biologically pure culture of the Bacillus licheniformis RTI184 deposited as ATCC PTA-121722, or a mutant thereof having all the identifying characteristics thereof to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit plant growth.

The growth benefit of the plant can be exhibited by one or a combination of improved seedling vigor, improved root development, improved plant growth, improved plant health, increased yield, or improved appearance.

The composition can be in the form of a liquid, an oil dispersion, a dust, a dry wettable powder, a spreadable granule, or a dry wettable granule. The Bacillus licheniformis RTI184 can be in the form of spores or in the form of vegetative cells. The Bacillus licheniformis RTI184 can be delivered at a rate of about 1.0×10⁸ CFU/ha to about 1.0×10¹³ CFU/ha to benefit the plant growth.

In the method, the composition can further include one or a combination of a microbial or a chemical insecticide, fungicide, nematicide, bacteriocide, herbicide, plant extract, or plant growth regulator, present in an amount suitable to benefit plant growth and/or to confer protection against pathogenic infection in the susceptible plant.

The method can further include applying a liquid fertilizer to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment of the present invention, a method is provided for benefiting plant growth, the method including: planting a seed of the plant or regenerating vegetative/callus tissue of the plant in a suitable growth medium, wherein the seed has been coated or the vegetative/callus tissue has been inoculated with a composition comprising a biologically pure culture of the Bacillus licheniformis RTI184 deposited as ATCC PTA-121722, or a mutant thereof having all the identifying characteristics thereof, wherein growth of the plant from the seed or the vegetative/callus tissue is benefited.

The growth benefit of the plant can be exhibited by one or a combination of improved seedling vigor, improved root development, improved plant growth, improved plant health, increased yield, or improved appearance.

The Bacillus licheniformis RTI184 can be in the form of spores. The Bacillus licheniformis RTI184 can be present in the form of spores at an amount ranging from about 1.0×10² CFU/seed to about 1.0×10⁹ CFU/seed. The composition coated on the seed can further comprise one or more of an insecticide, a fungicide, a nematicide, a bacteriocide, a plant growth regulatore or a fertilizer present in an amount suitable to benefit plant growth.

The growth benefit of the plant can be exhibited by one or a combination of improved seedling vigor, improved root development, improved plant growth, improved plant health, increased yield, improved appearance, or improved resistance to plant pathogens, or a combination thereof.

In one embodiment of the present invention, a method is provided for benefiting plant growth that includes delivering a combination of: a first composition comprising the biologically pure culture of the Bacillus licheniformis RTI184 deposited as ATCC No. PTA-121722, or mutants thereof having all the identifying characteristics thereof; and a second composition including a one or a combination of a microbial or a chemical insecticide, fungicide, nematicide, bacteriocide, herbicide, plant extract, plant growth regulator, or fertilizer to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, wherein each of the first and second compositions are delivered in an amount suitable for benefiting plant growth.

The method can further include applying a liquid fertilizer to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

The Bacillus licheniformis RTI184 can be in the form of spores or in the form of vegetative cells. The amount of Bacillus licheniformis RTI184 suitable for benefiting plant growth can range from about 1.0×10⁸ CFU/ha to about 1.0×10¹³ CFU/ha.

In one embodiment of the present invention, a method is provided for benefiting plant growth that includes: delivering a composition comprising: a biologically pure culture of the Bacillus licheniformis RTI184 deposited as ATCC No. PTA-121722, or a mutant thereof having all the identifying characteristics thereof; and one or a combination of a microbial or a chemical insecticide, fungicide, nematicide, bacteriocide, plant growth regulator, or fertilizer to: a seed of the plant, roots of the plant, a cutting of the plant, a graft of the plant, callus tissue of the plant; soil or growth medium surrounding the plant; soil or growth medium before sowing the seed of the plant in the soil or growth medium; or soil or growth medium before planting the plant, the plant cutting, the plant graft, or the plant callus tissue in the soil or growth medium, wherein each of the Bacillus licheniformis RTI184 and the one or a combination of a microbial or a chemical insecticide, fungicide, nematicide, bacteriocide, or plant growth regulator is present in an amount suitable for benefiting plant growth.

The plant growth benefit can be exhibited by improved seedling vigor, improved root development, improved plant health, increased plant mass, increased yield, improved appearance, improved resistance to plant pathogens, or a combination thereof.

The method can further include applying a liquid fertilizer to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

The Bacillus licheniformis RTI184 can be in the form of spores or in the form of vegetative cells. The amount of Bacillus licheniformis RTI184 suitable for benefiting plant growth can range from about 1.0×10⁸ CFU/ha to about 1.0×10¹³ CFU/ha.

In one embodiment of the present invention, a method is provided for benefiting plant rooting, the method including dipping a cutting of a plant in a composition and planting it in a suitable growth medium, wherein the composition comprises a biologically pure culture of a Bacillus licheniformis strain RTI184 deposited as ATCC PTA-121722, or a mutant thereof having all the identifying characteristics thereof, in an amount suitable to benefit plant rooting, wherein root formation and growth of the plant from the cutting is benefited.

The composition can be in the form of a liquid or a dry wettable powder. The Bacillus licheniformis RTI184 can be in the form of spores or vegetative cells. The composition can be in the form of a dry wettable powder and the Bacillus licheniformis RTI184 can be present in an amount of from about 1.0×10⁷ CFU/g to about 1.0×10⁹ CFU/g. The plant can be an ornamental plant. The plant can be a hydrangea.

In one embodiment of the present invention, a composition is provided, the composition comprising at least one of an isolated Fengycin MB-Cit compound and an isolated Dehydroxyfengycin MB-Cit compound in an amount suitable to confer one or both of a growth benefit on the plant or protection against a pathogenic infection in the susceptible plant, the Fengycin MB-Cit and Dehydroxyfengycin MB-Cit compounds having the formula:

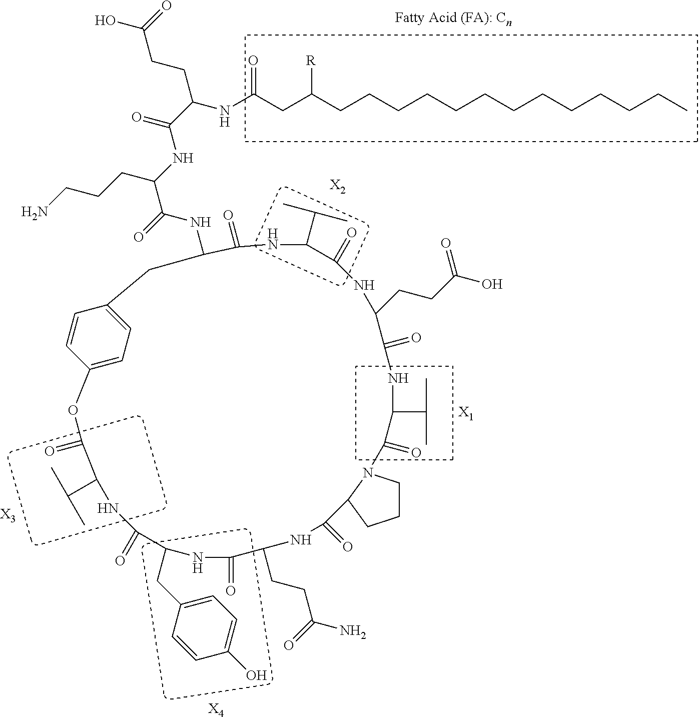

wherein n ranges from 8 to 20, FA is linear, iso, or anteiso, and R is OH, X₁ is Val, X₂ is Thr, X₃ is Met, and X₄ is Cit for Fengycin MB-Cit; and wherein n ranges from 8 to 20, FA is linear, iso, or anteiso, R is H, X₁ is Val, X₂ is Thr, X₃ is Met, and X₄ is Citruline for Dehydroxyfengycin MB-Cit. In another embodiment, the composition further comprises one or a combination of additional isolated Fengycin- and Dehydroxyfengycin-like compounds listed in Table VI in an amount suitable to confer one or both of a growth benefit on the plant or protection against a pathogenic infection in the susceptible plant.

The growth benefit of the plant and/or the conferred protection against pathogenic infection can be exhibited by improved seedling vigor, improved root development, improved plant growth, improved plant health, increased yield, improved appearance, improved resistance to plant pathogens, or reduced pathogenic infection, or a combination thereof.

The Fengycin MB-Cit compounds and Dehydroxyfengycin MB-Cit compounds and one or a combination of additional Fengycin- and Dehydroxyfengycin-like compounds can be isolated by first culturing the RTI184 Bacillus licheniformis strain, or another Bacillus licheniformis strain that produces the Fengycin MB-Cit and Dehydroxyfengycin MB-Cit compounds, under suitable conditions well known to those of skill in the art, such as, for example, those conditions described in the EXAMPLES herein, including, but not limited to, culturing the strain for 3 to 6 days in 869 or M2 media. The Fengycin-like and Dehydroxyfengycin-like cyclic lipopeptides present in the Bacillus licheniformis culture supernatant can then be further isolated using methods well known to those of skill in the art. For example, the Bacillus licheniformis culture supernatant can be acidified to pH 2 (Smyth, T J P et al., 2010, “Isolation and Analysis of Lipopeptides and High Molecular Weight Biosurfactants.” In: Handbook of Hydrocarbon and Lipid Microbiology, K. N. Timmis (Editor). pp 3687-3704), or treated with CaCl₂ (Ajesh, K et al., 2013, “Purification and characterization of antifungal lipopeptide from a soil isolated strain of Bacillus cereus.” In: Worldwide research efforts in the fighting against microbial pathogens: from basic research to technological developments. A. Mendez-Vilas (editor). pp: 227-231) or NH₄SO₄ (Kim, S H et al., 2000, Biotechnol Appl Biochem. 31 (Pt 3):249-253) with or without combining this with an organic extraction step (Kim, P I et al., 2004, J Appl Microbiol. 97(5): 942-949) such as various forms of phase separation including but not limited to direct liquid partitioning, membrane ultrafiltration, and foam fractionation (Baker, S C et al., 2010, Adv Exp Med Biol. 672:281-288).

In one embodiment, the Fengycin MB-Cit and the Dehydroxyfengycin MB-Cit compounds and the one or a combination of additional Fengycin- and Dehydroxyfengycin-like compounds listed in Table VI can be isolated from a biologically pure culture of a Bacillus licheniformis strain that can produce these compounds.

In one embodiment, the Fengycin MB-Cit and the Dehydroxyfengycin MB-Cit compounds and the one or a combination of additional Fengycin- and Dehydroxyfengycin-like compounds listed in Table VI can be isolated from a biologically pure culture of Bacillus licheniformis RTI184 deposited as ATCC No. PTA-121722.

In one embodiment, an extract is provided of a biologically pure culture of a Bacillus licheniformis strain, the extract including a Fengycin MB-Cit compound and a Dehydroxyfengycin MB-Cit compound and one or a combination of additional Fengycin- and Dehydroxyfengycin-like compounds listed in Table VI.

In one embodiment, an extract is provided of a biologically pure culture of Bacillus licheniformis RTI184 deposited as ATCC No. PTA-121722, the extract including a Fengycin MB-Cit compound and a Dehydroxyfengycin MB-Cit compound and one or a combination of additional Fengycin- and Dehydroxyfengycin-like compounds listed in Table VI.

The compositions including at least one of the isolated Fengycin MB-Cit and the Dehydroxyfengycin MB-Cit compounds and optionally one or a combination of additional isolated Fengycin- and Dehydroxyfengycin-like compounds can further include one or a combination of a microbial or a chemical insecticide, fungicide, nematicide, bacteriocide, herbicide, plant extract, or plant growth regulator, present in an amount suitable to benefit plant growth and/or to confer protection against pathogenic infection in the susceptible plant.

The compositions including the isolated Fengycin MB-Cit and the Dehydroxyfengycin MB-Cit compounds and one or a combination of additional isolated Fengycin- and Dehydroxyfengycin-like compounds can be in the form of a liquid, an oil dispersion, a dust, a spreadable granule, or a dry wettable granule.

In one embodiment, a method is provided for benefiting plant growth and/or conferring protection against a plant pathogenic infection that includes applying an effective amount of the extract or the composition comprising the isolated Fengycin MB-Cit and the Dehydroxyfengycin MB-Cit compounds and one or a combination of additional isolated Fengycin- and Dehydroxyfengycin-like compounds to the plant or fruit, or to the roots or soil around the roots of the plants to benefit the plant growth and/or conferring protection against the plant pathogenic infection. The growth benefit of the plant and/or the conferred protection can be exhibited by improved seedling vigor, improved root development, improved plant growth, improved plant health, increased yield, improved appearance, improved resistance to plant pathogens, or reduced pathogenic infection, or a combination thereof.

In the method for applying an effective amount of the extract or the composition comprising the isolated Fengycin MB-Cit and the Dehydroxyfengycin MB-Cit compounds and one or a combination of additional isolated Fengycin- and Dehydroxyfengycin-like compounds, the plant can include, for example, monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hardwood cuttings, Chestnuts, Oak, Maple, sugarcane, or sugarbeet.

In the method for applying an effective amount of the extract or the composition comprising the isolated Fengycin MB-Cit and the Dehydroxyfengycin MB-Cit compounds and one or a combination of additional isolated Fengycin- and Dehydroxyfengycin-like compounds, the pathogenic infection can be caused by a plant pathogen, including, for example, a plant fungal pathogen, a plant bacterial pathogen, a rust fungus a Botrytis spp., a Botrytis cinerea, a Botrytis squamosa, an Erwinia spp., an Erwinia carotovora, an Erwinia amylovora, a Dickeya spp., a Dickeya dadantii, a Dickeya solani, an Agrobacterium spp., a Agrobacterium tumefaciens, a Xanthomonas spp., a Xanthomonas axonopodis, a Xanthomonas campestris pv. carotae, a Xanthomonas pruni, a Xanthomonas arboricola, a Xanthomonas oryzae pv. oryzae, a Xylella spp., a Xylella fastidiosa, a Candidatus spp., a Candidatus liberibacter, a Fusarium spp., a Fusarium graminearum, a Fusarium oxysporum, a Fusarium oxysporum f. sp. Cubense, a Sclerotinia spp., a Sclerotinia sclerotiorum, a Sclerotinia minor, Sclerotinia homeocarpa, a Cercospora/Cercosporidium spp., an Uncinula spp., an Uncinula necator (Powdery Mildew), a Podosphaera spp. (Powdery Mildew), a Podosphaera leucotricha, a Podosphaera clandestine, a Phomopsis spp., a Phomopsis viticola, an Alternaria spp., an Alternaria tenuissima, an Alternaria porri, an Alternaria alternate, an Alternaria solani, an Alternaria tenuis, a Pseudomonas spp., a Pseudomonas syringae pv. Tomato, a Phytophthora spp., a Phytophthora infestans, a Phytophthora parasitica, a Phytophthora sojae, a Phytophthora capsici, a Phytophthora cinnamon, a Phytophthora fragariae, a Phytophthora spp., a Phytophthora ramorum, a Phytophthora palmivara, a Phytophthora nicotianae, a Phakopsora spp., a Phakopsora pachyrhizi, a Phakopsora meibomiae an Aspergillus spp., an Aspergillus flavus, an Aspergillus niger, a Uromyces spp., a Uromyces appendiculatus, a Cladosporium spp., a Cladosporium herbarum, a Rhizopus spp., a Rhizopus arrhizus, a Penicillium spp., a Rhizoctonia spp., a Rhizoctonia solani, a Rhizoctonia zeae, a Rhizoctonia oryzae, a Rhizoctonia caritae, a Rhizoctonia cerealis, a Rhizoctonia crocorum, a Rhizoctonia fragariae, a Rhizoctonia ramicola, a Rhizoctonia rubi, a Rhizoctonia leguminicola, a Macrophomina phaseolina, a Magnaorthe oryzae, a Mycosphaerella spp., Mycosphaerella graminocola, a Mycosphaerella fijiensis (Black sigatoga), a Mycosphaerella pomi, a Mycosphaerella citri, a Magnaporthe spp., a Magnaporthe grisea, a Monilinia spp., a Monilinia fruticola, a Monilinia vacciniicorymbosi, a Monilinia laxa, a Colletotrichum spp., a Colletotrichum gloeosporiodes, a Colletotrichum acutatum, a Colletotrichum Candidum, a Diaporthe spp., a Diaporthe citri, a Corynespora spp., a Corynespora Cassiicola, a Gymnosporangium spp., a Gymnosporangium juniperi-virginianae, a Schizothyrium spp., a Schizothyrium pomi, a Gloeodes spp., a Gloeodes pomigena, a Botryosphaeria spp., a Botryosphaeria dothidea, a Neofabraea spp., a Wilsonomyces spp., a Wilsonomyces carpophilus, a Sphaerotheca spp., a Sphaerotheca macularis, a Sphaerotheca pannosa, a Erysiphe spp., a Stagonospora spp., a Stagonospora nodorum, a Pythium spp., a Pythium ultimum, a Pythium aphanidermatum, a Pythium irregularum, a Pythium ulosum, a Pythium lutriarium, a Pythium sylvatium, a Venturia spp, a Venturia inaequalis, a Verticillium spp., a Ustilago spp., a Ustilago nuda, a Ustilago maydis, a Ustilago scitaminea, a Claviceps spp., a Claviceps puprrea, a Tilletia spp., a Tilletia tritici, a Tilletia laevis, a Tilletia horrid, a Tilletia controversa, a Phoma spp., a Phoma glycinicola, a Phoma exigua, a Phoma lingam, a Cocliobolus sativus, a Gaeumanomyces gaminis, a Colleototricum spp., a Rhychosporium spp., Rhychosporium secalis, a Biopolaris spp., a Helminthosporium spp., a Helminthosporium secalis, a Helminthosporium maydis, a Helminthosporium solai, and a Helminthosporium tritici-repentis, or combinations thereof.

### EXAMPLES

The following Examples have been included to provide guidance to one of ordinary skill in the art for practicing representative embodiments of the presently disclosed subject matter. In light of the present invention and the general level of skill in the art, those of skill can appreciate that the following Examples are intended to be exemplary only and that numerous changes, modifications, and alterations can be employed without departing from the scope of the presently disclosed subject matter.

### Example 1

Identification of a Bacterial Isolate as a Bacillus Licheniformis Through Sequence Analysis

A plant associated bacterial strain, designated herein as RTI184, was isolated from the root of rice grown in California. The 16S rRNA and the rpoB genes of the RTI184 strain were sequenced and subsequently compared to other known bacterial strains in the NCBI and RDP databases using BLAST. It was determined that the 16S RNA partial sequence of RTI184 (SEQ ID NO: 1) is nearly identical to the 16S rRNA gene sequence of two other known strains of B. licheniformis, Bacillus licheniformis strain 9945A (99%, 2 bp difference over 1545 bp in one copy of the 16S rRNA gene out of three different copies) and Bacillus licheniformis ATCC 14580 (99%, 8 bp difference over 1545 bp). In addition, it was determined that the rpoB sequence of RTI184 (SEQ ID NO: 2) has 100% sequence identity to known strain Bacillus licheniformis 9945A (CP005965) and 97% sequence identity to Bacillus licheniformis strain deposited as ATCC 14580 (97 bp difference over 3015 bp). To further discriminate between strain RTI184 and Bacillus licheniformis 9945A, the genome sequences for their pathways involved in biosynthesis of lichenysin, the characteristic anionic cyclic lipoheptapeptide biosurfactant produced by Bacillus licheniformis species, were compared. Although similar, some differences were observed between the lichA and lichB genes for strains RTI184 and 9945A. Thus, the RTI184 strain was identified as a unique strain of Bacillus licheniformis.

### Example 2

### Anti-Microbial Properties of Bacillus Licheniformis RTI184 Isolate

The antagonistic ability of the isolate against major plant pathogens was measured in plate assays. A plate assay for evaluation of antagonism against plant fungal pathogens was performed by growing the bacterial isolate and pathogenic fungi side by side on 869 agar plates at a distance of 4 cm. Plates were incubated at room temperature and checked regularly for up to two weeks for growth behaviors such as growth inhibition, niche occupation, or no effect. In the case of screening for antagonistic properties against bacterial pathogens, the pathogen was first spread as a lawn on 869 agar plates. Subsequently, 20 μl aliquots of a culture of RTI184 were spotted on the plate. Plates were incubated at room temperature and checked regularly for up to two weeks for an inhibition zone in the lawn around the positions were RTI184 had been applied. A summary of the antagonism activity is shown in Table I below.

TABLE I
Antagonistic properties of Bacillus licheniformis
RTI184 isolate against major plant pathogens

| Anti-Microbial Assays | RTI184 |
|---|---|
| Aspergillus flavus | ++ |
| Aspergillus nomius | + |
| Botrytis cinerea | +− |
| Fusarium graminearum | ++ |
| Fusarium oxysporum | + |
| Fusarium oxysporum spp. cubense | ++ |
| Magnaporthe grisea | + |
| Phytophthora capsici | +− |
| Pythium sylvatium | +− |
| Rhizoctonia solani | ++ |
| Pseudomonas syringae pv. tomato | − |
| Xanthomonas euvesicatoria | − |

+++ very strong activity,
++ strong activity,
+ activity,
+− weak activity,
− no activity observed

### Example 3

### Phenotypic Traits of Bacillus Licheniformis RTI184 Isolate

In addition to the antagonistic properties, various phenotypic traits were also measured for the Bacillus licheniformis RTI184 strain and the data are shown below in Table II. The assays were performed according to the procedures described in the text below Table II.

TABLE II
Phenotypic Assays: phytohormone production, acetoin and indole acetic
acid (IAA), and nutrient cycling of Bacillus licheniformis RTI184 isolate.

| Characteristic Assays | RTI184 |
|---|---|
| Acid production (Methyl Red) | − |
| Acetoin production (MR-VP) | +++ |
| Chitinase activity | + |
| Indole-3-Acetic Acid production | − |
| Protease activity | + |
| Phosphate solubilization | − |
| Phenotype | hard dry texture white/cream |

+++ very strong,
++ strong,
+ some,
+− weak,
− none observed

Acid and Acetoin Test.

20 μl of a starter culture in rich 869 media was transferred to 1 ml Methy Red-Voges Proskauer media (Sigma Aldrich 39484). Cultures were incubated for 2 days at 30° C. 200 rpm. 0.5 ml culture was transferred and 50 μl 0.2 g/l methyl red was added. Red color indicated acid production. The remaining 0.5 ml culture was mixed with 0.3 ml 5% alpha-napthol (Sigma Aldrich N1000) followed by 0.1 ml 40% KOH. Samples were interpreted after 30 minutes of incubation. Development of a red color indicated acetoin production. For both acid and acetoin tests non-inoculated media was used as a negative control (Sokol et al., 1979, Journal of Clinical Microbiology. 9: 538-540).

Indole-3-Acetic Acid.

20 μl of a starter culture in rich 869 media was transferred to 1 ml 1/10 869 Media supplemented with 0.5 g/l tryptophan (Sigma Aldrich T0254). Cultures were incubated for 4-5 days in the dark at 30° C., 200 RPM. Samples were centrifuged and 0.1 ml supernatant was mixed with 0.2 ml Salkowski's Reagent (35% perchloric acid, 10 mM FeCl3). After incubating for 30 minutes in the dark, samples resulting in pink color were recorded positive for IAA synthesis. Dilutions of IAA (Sigma Aldrich 15148) were used as a positive comparison; non inoculated media was used as negative control (Taghavi, et al., 2009, Applied and Environmental Microbiology 75: 748-757).

Phosphate Solubilizing Test.

Bacteria were plated on Pikovskaya (PVK) agar medium consisting of 10 g glucose, 5 g calcium triphosphate, 0.2 g potassium chloride, 0.5 g ammonium sulfate, 0.2 g sodium chloride, 0.1 g magnesium sulfate heptahydrate, 0.5 g yeast extract, 2 mg manganese sulfate, 2 mg iron sulfate and 15 g agar per liter, pH7, autoclaved. Zones of clearing were indicative of phosphate solubilizing bacteria (Sharma et al., 2011, Journal of Microbiology and Biotechnology Research 1: 90-95).

Chitinase Activity.

10% wet weight colloidal chitin was added to modified PVK agar medium (10 g glucose, 0.2 g potassium chloride, 0.5 g ammonium sulfate, 0.2 g sodium chloride, 0.1 g magnesium sulfate heptahydrate, 0.5 g yeast extract, 2 mg manganese sulfate, 2 mg iron sulfate and 15 g agar per liter, pH7, autoclaved). Bacteria were plated on these chitin plates; zones of clearing indicated chitinase activity (N. K. S. Murthy & Bleakley, 2012. “Simplified Method of Preparing Colloidal Chitin Used for Screening of Chitinase Producing Microorganisms”. The Internet Journal of Microbiology. 10(2)).

Protease Activity.

Bacteria were plated on 869 agar medium supplemented with 10% milk.

Clearing zones indicated the ability to break down proteins suggesting protease activity (Sokol et al., 1979, Journal of Clinical Microbiology. 9: 538-540).

### Example 4

### Effect of Bacillus Licheniformis RTI184 on Seed Germination, Root Development and Architecture

Experiments were performed to determine the effects of application of the B. licheniformis RTI184 strain to seed on seed germination and root development and architecture. Experiments were performed as described below using both vegetative cells and spores of RTI184.

Vegetative Cells:

Assays with vegetative cells of RTI184 were performed using seed from corn and soybean. RTI184 was plated onto 869 media from a frozen stock and grown overnight at 30° C. An isolated colony was taken from the plate and inoculated into a 50 mL conical tube containing 20 mL of 869 broth. The culture was incubated overnight with shaking at 30° C. and 200 RPM. The overnight culture was centrifuged at 10,000 RPM for 10 minutes. Supernatant was discarded and pellet was resuspended in MgSO₄ to wash. The mixture was centrifuged again for 10 minutes at 10,000 RPM. The supernatant was discarded and the pellet was resuspended in Modified Hoagland's solution. The mixture was then diluted to provide an initial concentration (10⁰). From this 10⁻¹, 10⁻², 10⁻³, 10⁻⁴ and 10⁻³ dilutions of the RTI184 culture were made. For the seed germination experiments for each type of seed, 100 mm petri dishes were labeled with RTI184 or control, the dilution, and the date. A sterile filter paper was placed in the bottom of each dish. Five (5) to eight (8) seeds were placed in a single petri dish depending on the type of seed (e.g., larger seeds such as corn had smaller numbers of seed/plate). Five mL of each dilution of RTI184 was added to the plates and the seeds were incubated at 21° C. Control plates contained seeds and Modified Hoagland's solution without added bacteria. Images of the plates were taken after 4 and 7 days. Sterile DI water was added to the plates when they began to dry out. Corn and soybean data are shown in Table III below. In addition, FIGS. 1A-1D are images of soybean seeds showing the positive effects on root hair development after inoculation by vegetative cells of RTI184 diluted by 10° (B), 10⁻¹(C), and 10⁻²(D), corresponding to (B) 2.62×10⁸ CFU/ml, (C) 2.62×10² CFU/ml, and (D) 2.62×10⁶ CFU/ml, respectively, after 7 days of growth as compared to untreated control (A). The data show that addition of the RTI184 cells stimulated formation of fine root hairs compared to uninoculated control seeds. Fine root hairs are important in the uptake of water, nutrients and plant interaction with other microorganisms in the rhizosphere.

TABLE III
Seed germination assay for treatment with vegetative cells of RTI184

| Seed Germination Assays—RTI184 | | | | |
|---|---|---|---|---|
| (Vegetative cells) | | Dilution | | |
| Crop | Starting CFU/ml | 10⁰ | 10⁻¹ | 10⁻² |
| Corn | 2.62 × 10⁸ cfu/ml | = | = | ND |
| Soy | 2.62 × 10⁸ cfu/ml | ++ | +++ | +++ |

+++ very pronounced growth benefit,
++ strong growth benefit,
+ growth benefit,
+− weak growth benefit,
= no effect observed,
ND not determined.

Spores:

For the experiments using spores of RTI184, the strain was sporulated in 2XSG in a 14 L fermenter. Spores were collected but not washed afterwards at a concentration of at least 1.0×10² CFU/mL. The spore concentration was diluted down by a factor of 10 or greater in the experiments. Experiments were performed with seeds of cucumber, pepper, tomato, radish, squash, grass (Kentucky Bluegrass), and marigold. A sterile filter paper was placed in the bottom of each sterile plastic growth chamber, and ten seeds were placed in each container. Three mL of each dilution of RTI184 spores was added to the growth chambers, which were closed and incubated at 19° C. for 7 days, after which the seedlings were imaged. Data are shown in Table IV below. Inhibition of seed germination and growth was not observed for treatment with RTI184 for any of the plant species compared to non-inoculated controls. In addition, images of the positive effects of inoculation of seed with the RTI184 strain on MONEY MAKER Tomato are shown in FIGS. 2A-2B. Control plants are shown in FIG. 2A and plants inoculated with RTI184 are shown in FIG. 2B.

TABLE IV
Seed germination assay for treatment with spores of RTI184

| Seed Germination Assays—RTI184 | | Dilution | | |
|---|---|---|---|---|
| Crop | Starting CFU/ml | 10⁰ | 10⁻¹ | 10⁻² |
| Cucumber | 2.68 × 10⁸ cfu/mL | + | ++ | + |
| Squash | 2.68 × 10⁸ cfu/mL | = | = | = |
| Kentucky Bluegrass | 2.68 × 10⁸ cfu/mL | = | = | = |
| Marigold | 1.00 × 10⁷ cfu/mL | + | + | + |
| Pepper | 2.68 × 10⁸ cfu/mL | ND | ND | + |
| Radish | 1.00 × 10⁷ cfu/mL | = | = | = |
| Tomato—BETTER BOY | 1.00 × 10⁷ cfu/mL | + | + | = |
| Tomato—MONEY MAKER | 2.68 × 10⁸ cfu/mL | +++ | ++ | +++ |

+++ very pronounced growth benefit,
++ strong growth benefit,
+ growth benefit,
+− weak growth benefit,
= no effect observed,
ND not determined

### Example 5

### Growth Effects of Bacillus Licheniformis Isolate RTI184 in Corn-Seed Inoculation

The effect of application of the bacterial isolate RTI184 on growth and vigor for corn was determined. The experiment was performed by inoculating surface sterilized germinated corn seeds for 2 days in a suspension of 10⁷ CFU/ml of the bacterium at room temperature under shaking. Subsequently, the inoculated seeds were planted in 1 gallon pots filled with PROMIX BX (PREMIER TECH, INC; Quebec, Canada) which was limed to a pH of 6.5. For each treatment 9 pots were seeded with a single corn seed planted at 5 cm depth. Pots were incubated in the greenhouse at 22° C. with light and dark cycle of 14/10 hrs and watered twice a week as needed. After 8 weeks, plants were harvested and their height, fresh, and dry weight were measured and compared to data obtained for non-inoculated control plants. Dry weight was determined as a total weight per 9 plants resulting in a total average dry plant weight equal to 14.09 g for the plants inoculated with the Bacillus licheniformis RTI184 strain versus a weight equal to 11.24 g for the non-inoculated control which is a 25% increase in dry weight over the non-inoculated control. In addition, FIGS. 3A-3B show photographic images of plants inoculated with Bacillus licheniformis RTI184 (FIG. 3A) as compared to control plants (FIG. 3B).

### Example 6

### Growth Effects of Bacillus Licheniformis Isolate RTI184 in Cucumber, Tomato, and Pepper-PROMIX BX Potting Soil Enhanced with RTI184 Spores

The effect of application of the bacterial isolate RTI184 on growth and vigor for cucumber, tomato, and pepper was determined. In this experiment, cucumber, tomato and pepper seeds were planted in PROMIX BX (PREMIER TECH, INC; Quebec, Canada) potting soil, limed to a pH of 6.5 and enhanced with 1×10⁷ spores/g Bacillus licheniformis strain RTI184. Seeds were planted in the RTI184-enhanced PROMIX BX (PREMIER TECH, INC; Quebec, Canada) soil in 6″ pots. One seed was planted per pot and there were 8 replicates per treatment. Plants were harvested and their dry shoot weight was measured and compared to data obtained for non-inoculated control plants. Dry shoot biomass was determined as a total weight per 8 plants. The data are shown below in Table V and show that RTI184 outperformed the control for all crop types.

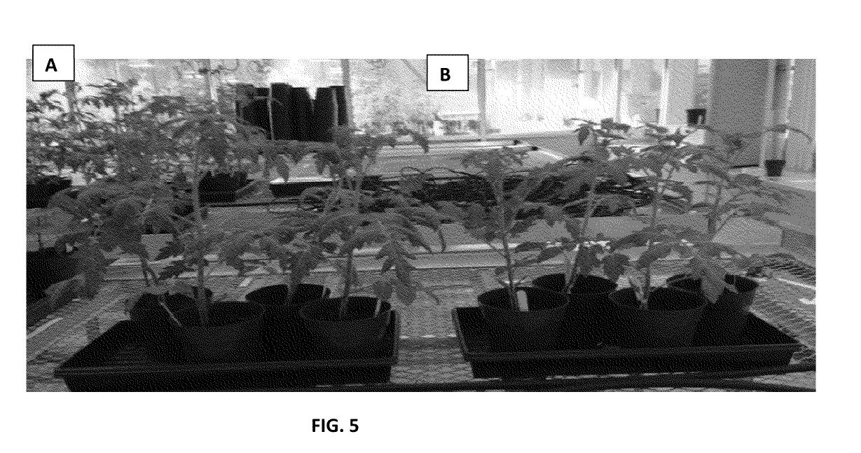

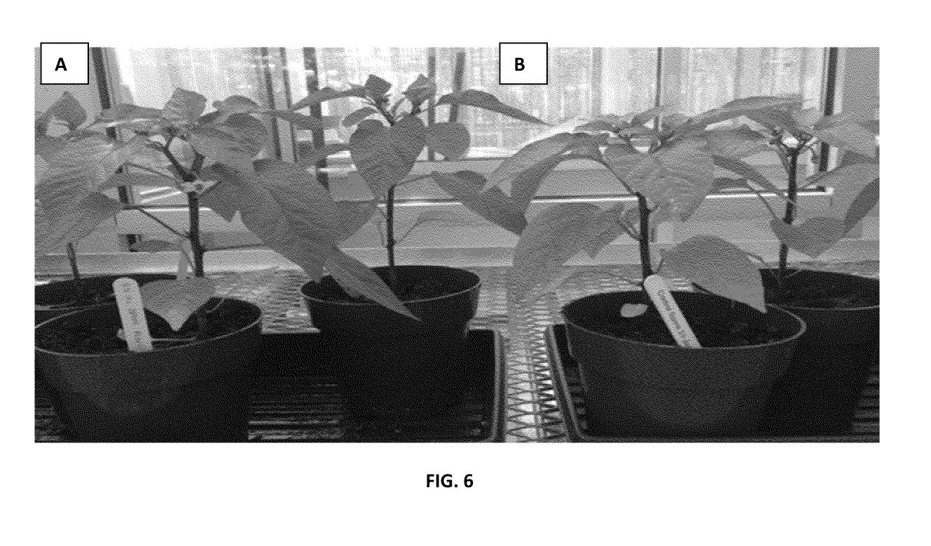

In addition, FIGS. 4A-4B are images of the cucumber data showing the positive effects on growth and vigor in cucumber after planting in the RTI184-enhanced soil: A) control cucumber plants; and B) cucumber plants grown in Bacillus licheniformis RTI184-enhanced soil. FIGS. 5A-5B are images of the tomato data showing the positive effects on growth and vigor in tomato after planting in the RTI184-enhanced soil: A) tomato plants grown in Bacillus licheniformis RTI184-enhanced soil; and B) control tomato plants. FIGS. 6A-6B are images of the pepper data showing the positive effects on growth and vigor in pepper after planting in the RTI184-enhanced soil: A) pepper plants grown in Bacillus licheniformis RTI184-enhanced soil; and B) control pepper plants.

TABLE V
Effect on dry shoot mass in cucumber, tomato, and pepper after
growth in PROMIX BX potting soil containing 1 × 10⁷ spores/g
Bacillus licheniformis strain RTI184.

| | Dry Weight of Shoot | % Increase |
|---|---|---|
| Crop | Biomass (gram) | Over Control |
| Cucumber—Control | 5.09 | |
| Cucumber | 7.33 | 44% |
| Tomato—Control | 5.55 | |
| Tomato | 9.32 | 68% |
| Pepper—Control | 2.20 | |
| Pepper | 2.77 | 26% |

### Example 7

### Identification of New Metabolites Produced by Bacillus Licheniformis RTI184 Isolate

It has been previously reported that five classes of Fengycin-type metabolites and Dehydroxyfengycin-type metabolites are produced by microbial species including Bacillus licheniformis (Li, Xing-Yu, et al., 2013, J. Microbiol. Biotechnol. 23(3), 313-321; Pecci Y, et al. 2010 Mass Spectrom., 45(7):772-77.)

These metabolites, belonging to the class of cyclic lipopeptides, are cyclic peptide molecules that also contain a fatty acid group. The five classes of Fengycin- and Dehydroxyfengycin-type metabolites are referred to as A, B, C, D and S. The backbone structure of these metabolites as well as the specific amino acid sequence for each of the five classes is shown in FIG. 7. The Fengycin- and Dehydroxyfengycin-type metabolites produced by Bacillus licheniformis strain RTI184 were analyzed using UHPLC-TOF MS. The molecular weights of the Fengycin-type metabolites produced by the RTI184 strain after both 3 and 6 days growth in rich media (either in 869 or in M2 medium) at 30° C. were compared to the theoretical molecular weights expected for the Fengycin- and Dehydroxyfengycin-type metabolites. In addition, to determine the amino acid composition of the various Fengycin-type metabolites produced by the RTI184 strain, peptide sequencing using LC-MS-MS was performed on each of the Fengycin-type metabolites previously identified via UHPLC-TOF MS. These data are shown in Table VI below. In this manner, it was determined that Bacillus licheniformis strain RTI184 did not produce Fengycin A, B, C, D, or S.

Surprisingly, it was determined that the RTI184 strain produces previously unidentified derivatives of these compounds where the L-isoleucine at position 8 of the cyclic peptide chain (referred to as X₃ in FIG. 7) is replaced by L-methionine. The new classes of Fengycin and Dehydroxyfengycin are referred to herein as MA, MB and MC, referring to derivatives of classes A, B and C in which the L-isoleucine at X₃ in FIG. 7 has been replaced by L-methionine. The newly identified molecules are shown in bold in FIG. 7 and in Table VI.

In addition to these new derivatives, another previously unidentified class produced by the Bacillus licheniformis strain RTI184 was identified, in which the Tyrosine (Tyr) of Fengycin MB and Dehydroxyfengycin MB (position X₄ in FIG. 7) is replaced by the α-amino acid, Citruline. This new class of Fengycin and Dehydroxyfengycin is being referred to herein as Fengycin MB-Cit and Dehydroxyfengycin MB-Cit and is shown in bold in FIG. 7 and in Table VI.

It was further determined that the Bacillus licheniformis strain RTI184 produces an additional class of Fengycin and Dehydroxyfengycin that has not been previously identified. In this class, the L-isoleucine of Fengycin B and Dehydroxyfengycin B (position X₃ in FIG. 7) is replaced by L-homo-cysteine (Hcy). These previously unidentified Fengycin and Dehydroxyfengycin metabolites are referred to herein as Fengycin H and Dehydroxyfengycin H and are shown in bold in FIG. 7 and in Table VI.

It was further determined that the Bacillus licheniformis strain RTI184 produces an additional class of Dehydroxyfengycin that has not been previously reported. In this class, position X₁ in FIG. 7 is replaced by L-isoleucine. This previously unreported Dehydroxyfengycin metabolite is referred to herein as Dehydroxyfengycin I and is shown in bold in FIG. 7 and in Table VI.

A summary of the amino acid sequences for the previously reported Fengycin- and Dehydroxyfengycin-type lipopeptides and the newly identified metabolites (shown in bold) is provided in Table VI below.

TABLE VI
Summary of MS/MS identification of Fengycin-type metabolites in Bacillus licheniformis RTI184 isolate.

| | | | | | | Ring | Theoretical C16 | Theoretical | Observed |
|---|---|---|---|---|---|---|---|---|---|
| Homolog | X₁ | X₂ | X₃ | X₄ | R | Mass | Molecular Formula | C16 [M + H]⁺ | RTI184 |
| Fengycin A | Ala | Thr | Ile | Tyr | OH | 1080.6 | C₇₂H₁₁₀N₁₂O₂₀ | 1463.8 | Not observed |
| Fengycin B | Val | Thr | Ile | Tyr | OH | 1108.7 | C₇₄H₁₁₄N₁₂O₂₀ | 1491.8 | Not observed |
| Fengycin C | Aba | Thr | Ile | Tyr | OH | 1094.6 | C₇₃H₁₁₂N₁₂O₂₀ | 1477.8 | Not observed |
| Fengycin D | Val | Thr | Val | Tyr | OH | 1094.6 | C₇₃H₁₁₂N₁₂O₂₀ | 1477.8 | Not observed |
| Fengycin S | Val | Ser | Ile | Tyr | OH | 1094.6 | C₇₃H₁₁₂N₁₂O₂₀ | 1477.8 | Not observed |
| Fengycin I | Ile | Thr | Ile | Tyr | OH | 1122.8 | C₇₇H₁₁₆N₁₂O₂₀ | 1505.8 | Not observed |
| Fengycin MA | Ala | Thr | Met | Tyr | OH | 1098.7 | C₇₁H₁₀₈N₁₂O₂₀S | 1481.8 | C17 |
| Fengycin MB | Val | Thr | Met | Tyr | OH | 1126.8 | C₇₃H₁₁₂N₁₂O₂₀S | 1509.8 | C15, C17 |
| Fengycin MC | Aba | Thr | Met | Tyr | OH | 1112.7 | C₇₂H₁₁₀N₁₂O₂₀S | 1495.8 | C16 |
| Fengycin H | Val | Thr | Hcy | Tyr | OH | 1112.7 | C₇₂H₁₁₀N₁₂O₂₀S | 1495.8 | C16 |
| Dehydroxyfengycin A | Ala | Thr | Ile | Tyr | H | 1080.6 | C₇₂H₁₁₀N₁₂O₁₉ | 1447.8 | C14 |
| Dehydroxyfengycin B | Val | Thr | Ile | Tyr | H | 1108.7 | C₇₄H₁₁₄N₁₂O₁₉ | 1475.8 | C17 |
| Dehydroxyfengycin C | Aba | Thr | Ile | Tyr | H | 1094.6 | C₇₃H₁₁₂N₁₂O₁₉ | 1461.8 | Not observed |
| Dehydroxyfengycin D | Val | Thr | Val | Tyr | H | 1094.6 | C₇₃H₁₁₂N₁₂O₁₉ | 1461.8 | Not observed |
| Dehydroxyfengycin S | Val | Ser | Ile | Tyr | H | 1094.6 | C₇₃H₁₁₂N₁₂O₁₉ | 1461.8 | Not observed |
| Dehydroxyfengycin I | Ile | Thr | Ile | Tyr | H | 1122.8 | C₇₅H₁₁₆N₁₂O₁₉ | 1489.9 | C16 |
| Dehydroxyfengycin MA | Ala | Thr | Met | Tyr | H | 1098.7 | C₇₁H₁₀₈N₁₂O₁₉S | 1465.8 | C14, C17 |
| Dehydroxyfengycin MB | Val | Thr | Met | Tyr | H | 1126.8 | C₇₃H₁₁₂N₁₂O₁₉S | 1493.8 | C15, C16 |
| Dehydroxyfengycin MC | Aba | Thr | Met | Tyr | H | 1112.7 | C₇₂H₁₁₀N₁₂O₁₉S | 1479.8 | C16, C17 |
| Dehydroxyfengycin H | Val | Thr | Hcy | Tyr | H | 1112.7 | C₇₂H₁₁₀N₁₂O₁₉S | 1479.8 | C16, C17 |
| Fengycin MB-Cit | Val | Thr | Met | Cit | OH | 1120.6 | C₇₀H₁₁₄N₁₄O₂₀S | 1503.8 | C15, C17 |
| Dehydroxyfengycin MB- | Val | Thr | Met | Cit | H | 1120.6 | C₇₀H₁₁₄N₁₄O₁₉S | 1487.8 | C15, C16, |
| Cit | | | | | | | | | C17 |

To determine whether the synthesis of the newly identified types of Fengycin- and Dehydroxyfengycin-type metabolites is intrinsic to the species Bacillus licheniformis or is instead specific to individual strains of Bacillus licheniformis, the synthesis of these types of molecules was compared between ten Bacillus licheniformis strains. The ten bacterial strains selected for this analysis were identified as being Bacillus licheniformis strains based on sequence comparison of their highly conserved 16S rRNA and rpoB gene sequences. The genomic DNA of each strain was isolated and compared by BOX-PCR pattern using a previously described method (Vinuesa, P. et al., 1998, Applied and Environmental Microbiology, 64, 2096-2104) and an image of the gel showing the resulting BOX-PCR patterns for the strains is shown in FIG. 8. Specifically, FIG. 8 shows agarose gel electrophoresis of BOX-PCR fingerprinting patterns for genomic DNA of Bacillus licheniformis strains CH200 deposited as Accession No. DSM 17236, RTI1242, RTI1249, RTI184, RTI1243, RTI1112, FCC1598, RTI239, RTI241, and RTI253. As molecular size marker, the 1 kb DNA ladder (FERMENTAS) was used. Based on their BOX-PCR pattern, the ten strains fell into three main groups, Group 1, Group 2A-2B (Group 2A and 2B represent the position on the gel in FIG. 8), and Group 3, which comprises the strains not belonging to the Groups 1 and 2.

To determine the type of Fengycin- and Dehydroxyfengycin-type metabolites produced by each of the ten Bacillus licheniformis strains, the strains were analyzed using UHPLC-TOF MS. In addition, the Lichenysin-type metabolites, characteristic for Bacillus licheniformis, were also analyzed as internal control. The results of the UHPLC-TOF MS analysis are summarized in Table VII below. The lichenysin and fengycin-type and dehyroxyfengycin-type molecules, their lipid modification (fatty acid (FA) chain length), predicted molecular mass, and their presence or absence in the culture supernatant of each of the ten Bacillus licheniformis strains grown for 6 days in M2 media are presented in Table VII. The data show that the Lichenysin-type metabolites were synthesized by all ten strains, confirming that they are true Bacillus licheniformis strains. On the other hand, major differences were observed between the ten strains with regard to the production of the Fengycin- and Dehydroxyfengycin-type metabolites.

TABLE VII
Summary of UHPLC-TOF MS identification of Fengycin-type and Dehydroxyfengycin-type
metabolites in 10 different Bacillus licheniformis isolates.

| | FA | | | | | | | | | | | |
|---|---|---|---|---|---|---|---|---|---|---|---|---|
| Compound | chain | [M + H]⁺ | CH200 | RTI1242 | RTI1249 | RTI184 | RTI1243 | RTI1112 | FCC1598 | RTI1239 | RTI1241 | RTI1253 |
| dehydroxy | C16- | 1461.8239- | + | − | − | + | − | + | + | + | − | − |
| Fengycin | C18 | 1489.8552 | | | | | | | | | | |
| A/B/C/D/I/S | | | | | | | | | | | | |
| dehydroxy | C15- | 1437.7334- | + | − | − | + | − | + | + | + | − | − |
| Fengycin | C17 | 1493.796 | | | | | | | | | | |
| H/MA/MB/ | | | | | | | | | | | | |
| MC | | | | | | | | | | | | |
| dehydroxy- | C15- | 1473.8021- | + | − | − | + | − | + | + | + | − | − |
| fengycin | C17 | 1501.8334 | | | | | | | | | | |
| MB-Cit | | | | | | | | | | | | |
| Fengycin | C15- | 1477.8188- | + | − | − | − | − | − | + | + | − | − |
| A/B/C/D/I/S | C17 | 1491.8344 | | | | | | | | | | |
| Fengycin | C14- | 1481.7596- | + | − | − | + | − | + | − | + | − | − |
| H/MA/MB/ | C17 | 1495.7752 | | | | | | | | | | |
| MC | | | | | | | | | | | | |
| Fengycin | C15- | 1489.797- | + | − | − | + | − | + | + | + | − | − |
| MB-Cit | C17 | 1517.8283 | | | | | | | | | | |
| Lichenysin | C12- | 993.6594- | + | + | + | + | + | + | + | + | + | + |
| A/D1/D2/D3/ | C17 | 1063.7376 | | | | | | | | | | |
| G1/G2/G3/ | | | | | | | | | | | | |
| G4/G5 | | | | | | | | | | | | |

Strains RTI184 and RTI1112 (Group 2), which had identical BOX-PCR patterns, were found to produce the same type of Fengycin- and Dehydroxyfengycin-type metabolites, including dehydroxy Fengycin A/B/C/D/I/S, dehydroxy Fengycin H/MA/MB/MC, dehydroxyfengycin MB-Cit, Fengycin H/MA/MB/MC and Fengycin MB-Cit, but failed to produce the Fengycin A/B/C/D/I/S type metabolites. On the other hand, strain FCC1598 which also falls into Group 2, produced the Fengycin A/B/C/D/I/S type metabolites, but failed to produce the Fengycin H/MA/MB/MC-type metabolites. Surprisingly, strain RTI1243, which also belongs to Group 2, did not produce any of the Fengycin- and Dehydroxyfengycin-type metabolites. Finally, two of the strains belonging to Group 1 (RTI1242 and RTI1249) and two strains belonging to Group 3 (RTI1241 and RTI1253) failed to produce any of the Fengycin- and Dehydroxyfengycin-type metabolites, whereas the CH200 and RTI1239, belonging to Group 1 and Group 3, respectively, produced all of the Fengycin- and Dehydroxyfengycin-type metabolites. Based on these results, it was concluded that the synthesis of the different types of Fengycin- and Dehydroxyfengycin-type metabolites, including the newly identified citruline-containing metabolites, is strain dependent rather than intrinsic to the species Bacillus licheniformis. For example, even closely related Bacillus licheniformis Group 2 strains produced different Fengycin- and Dehydroxyfengycin-type molecules and one closely related Group 2 strain failed to produce any Fengycin- or Dehydroxyfengycin-type metabolites at all.

### Example 8

### Effects of Drip Irrigation with Bacillus Licheniformis Isolate RTI184 on Squash, Broccoli, Turnip, Lettuce and Strawberry

Experiments were performed to determine the effect of drip irrigation with spores of the B. licheniformis RTI184 strain on squash, broccoli, turnip, and strawberry. The effects on plant yield were determined according to the experiments described below.

A field trial was performed for squash plants where drip irrigation was used to apply 1.5×10¹¹, 2.5×10¹², or 2.5×10¹³ CFU/hectare of B. licheniformis RTI184 spores at the time of planting, and again 2 weeks later. As compared to control plants in which B. licheniformis RTI184 spores were not included in the irrigation, addition of the RTI184 spores at all concentrations resulted in an increase in yield for both total and marketable squash. Specifically, RTI184 treated plants (application rate 2.5×10¹² CFU/hectare) resulted in an average of 33 kg of total squash of which 26 kg was marketable, as compared to 22 kg of total squash of which 17 kg was marketable for the untreated control plants. This is a 50% increase in weight of total squash and a 53% increase in weight of marketable squash. The substantial increase in both total squash weight and marketable squash weight of the plants treated with RTI184 relative to the control plants demonstrates the positive growth effect provided by treatment with the RTI184 spores.

A similar field trial was performed in which broccoli plants were drip irrigated with 1.5×10¹¹, 2.5×10¹², or 2.5×10¹³ CFU/hectare of B. licheniformis RTI184 spores at the time of planting and again 2 weeks later. As compared to control plants in which B. licheniformis RTI184 spores were not included in the irrigation, addition of the RTI184 spores resulted in a consistent increase in fresh weight yield from 3 kg (control) to 4 kg (2.5×10¹³ CFU/hectare RTI184), 3.9 kg (2.5×10¹² CFU/hectare RTI184), and 4.6 kg (1.5×10¹¹ CFU/hectare RTI184) or a 33%, 30%, and 53% increase in weight, respectively. The substantial increase in fresh weight of the plants treated with RTI184 relative to the control plants demonstrates the positive growth effect provided by treatment with the RTI184 spores.

A similar field trial was performed in which turnip plants were drip irrigated with 1.5×10¹¹, 2.5×10¹², or 2.5×10¹³ CFU/hectare of B. licheniformis RTI184 spores at the time of planting and again 2 weeks later. As compared to control plants in which B. licheniformis RTI184 spores were not included in the irrigation, addition of the RTI184 spores at all concentrations resulted in a consistent increase in tuber weight yield from 3.3 kgs (control) to approximately 5.3 kgs which is a 60% increase. The substantial increase in tuber weight of the plants treated with RTI184 relative to the control plants demonstrates the positive growth effect provided by treatment with the RTI184 spores.

A similar field trial was performed in which lettuce plants were drip irrigated with 12.5×10¹² CFU/hectare of B. licheniformis RTI184 spores at the time of planting and again 2 weeks later. As compared to control plants in which B. licheniformis RTI184 spores were not included in the irrigation, addition of the RTI184 spores resulted in a consistent increase in lettuce weight yield from 45.6 kgs (control) to 52.8 kgs, which is a 16% increase. The increased weight of the plants treated with RTI184 relative to the control plants demonstrates the positive growth effect provided by treatment with the RTI184 spores.

A similar field trial was performed in which strawberry plants were drip irrigated with 1.5×10¹¹, 2.5×10¹², or 2.5×10¹³ CFU/hectare of B. licheniformis RTI184 spores at the time of planting and again 2 weeks later. As compared to control plants in which B. licheniformis RTI184 spores were not included in the irrigation, addition of the RTI184 spores resulted in a increase in total yield of 5% (1.5×10¹¹ CFU/hectare RTI184), 8% (2.5×10¹² CFU/hectare RTI184), and 11% (2.5×10¹³ CFU/hectare RTI184). The increase in yield of the plants treated with RTI184 relative to the control plants demonstrates the positive growth effect provided by treatment with the RTI184 spores.

### Example 9

### Growth Effects of Bacillus Licheniformis Isolate RTI184 on Potato Plants Grown in Nematode-Infected Soil

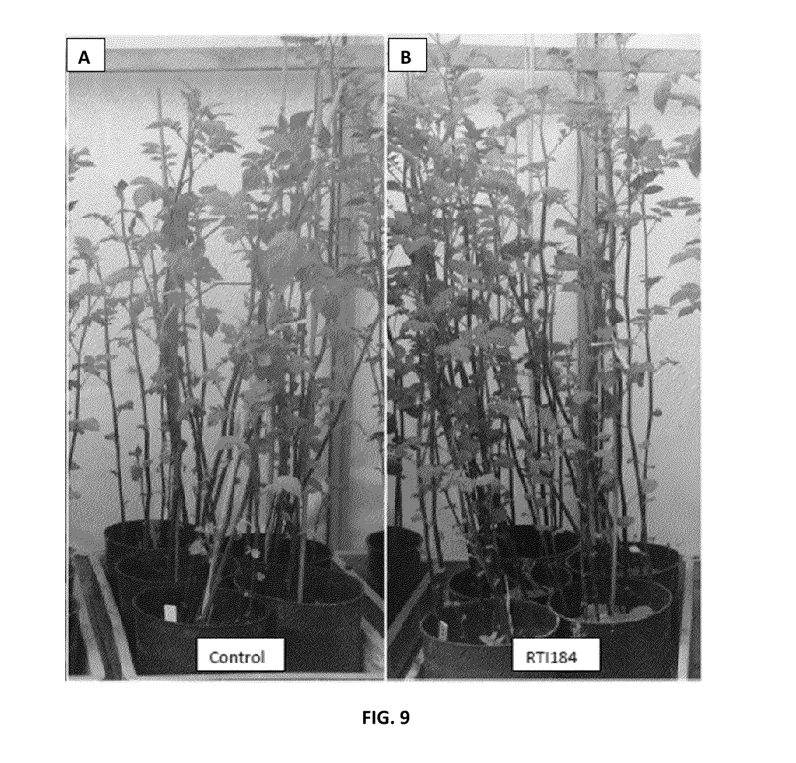

In this experiment, the effect of application of the bacterial isolate RTI184 on growth and vigor for potato plants grown in nematode infected soil (Globedera sp., approximately 1750 live eggs and juveniles per 100 ml soil) was determined. Potatos (variety “Bintje”) were planted in soil infected with Globodera sp. and enhanced with or drip irrigated with 10E⁺⁹ cfu spores per liter soil of Bacillus licheniformis strain RTI184. Images of the plants after 48 days of growth in a greenhouse are shown in FIGS. 9A-9B. FIG. 9A shows the control plants that were not treated with the RTI184 spores and FIG. 9B shows the plants treated with RTI184. The increased size of the plants treated with RTI1184 relative to the control plants demonstrates the positive growth effect provided by treatment with the RTI184 spores.

### REFERENCES

All publications, patent applications, patents, and other references cited herein are incorporated hereinby reference in their entireties.

That which is claimed:

1. A composition for benefiting plant growth, the composition comprising a biologically pure culture of Bacillus licheniformis RTI184 deposited as ATCC No. PTA-121722, or a mutant thereof having all the identifying characteristics thereof, present in an amount suitable to benefit plant growth.

2. The composition of claim 1, wherein the composition is capable of benefiting plant growth when applied to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

3. The composition of claim 1, wherein the growth benefit of the plant is exhibited by improved seedling vigor, improved root development, improved plant growth, improved plant health, increased yield, or improved appearance, or a combination thereof.

4. The composition of claim 1, wherein the composition is in the form of a liquid, an oil dispersion, a dust, a dry wettable powder, a spreadable granule, or a dry wettable granule.

5. The composition of claim 1, wherein the composition is in the form of a liquid and the Bacillus licheniformis RTI184 is present at a concentration of from about 1.0×10⁹ CFU/ml to about 1.0×10¹² CFU/ml.

6. The composition of claim 1, wherein the composition is in the form of a dust, a dry wettable powder, a spreadable granule, or a dry wettable granule and the Bacillus licheniformis RTI184 is present in an amount of from about 1.0×10⁹ CFU/g to about 1.0×10¹² CFU/g.

7. The composition of claim 1, wherein the composition is in the form of an oil dispersion and the Bacillus licheniformis RTI184 is present at a concentration of from about 1.0×10⁹ CFU/ml to about 1.0×10¹² CFU/ml.

8. The composition of claim 1, wherein the Bacillus licheniformis RTI184 is in the form of spores.

9. The composition of claim 1, wherein the Bacillus licheniformis RTI184 is in the form of vegetative cells.

10. The composition of claim 1, further comprising one or a combination of a microbial or a chemical insecticide, fungicide, nematicide, bacteriocide, herbicide, plant extract, or plant growth regulator present in an amount suitable to benefit plant growth and/or to confer protection against a pathogenic infection in a susceptible plant.

11. The composition of claim 10, wherein the insecticide comprises bifenthrin.

12. The composition of claim 10, wherein the nematicide comprises cadusafos.

13. The composition of claim 10, wherein the insecticide comprises bifenthrin and clothianidin.

14. The composition of claim 10, formulated as a liquid.

15. The composition of claim 14, wherein the insecticide comprises bifenthrin or zeta-cypermethrin.

16. The composition of claim 1, wherein the composition is in the form of a planting matrix.

17. The composition of claim 16, wherein the planting matrix is in the form of a potting soil.

18. The composition of claim 1, wherein the plant comprises monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

19. A plant seed coated with a composition comprising:
spores of a biologically pure culture of Bacillus licheniformis RTI184 deposited as ATCC No. PTA-121722, or a mutant thereof having all the identifying characteristics thereof, present in an amount suitable to benefit plant growth.

20. The plant seed of claim 19, wherein the composition comprises an amount of Bacillus licheniformis spores from about 1.0×10² CFU/seed to about 1.0×10⁹ CFU/seed.

21. The plant seed of claim 19, wherein the seed comprises the seed of monocots, dicots, Cereals, Corn, Sweet Corn, Popcorn, Seed Corn, Silage Corn, Field Corn, Rice, Wheat, Barley, Sorghum, Brassica Vegetables, Broccoli, Cabbage, Cauliflower, Brussels Sprouts, Collards, Kale, Mustard Greens, Kohlrabi, Bulb Vegetables, Onion, Garlic, Shallots, Fruiting Vegetables, Pepper, Tomato, Eggplant, Ground Cherry, Tomatillo, Okra, Grape, Herbs/Spices, Cucurbit Vegetables, Cucumber, Cantaloupe, Melon, Muskmelon, Squash, Watermelon, Pumpkin, Eggplant, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Cotton, Flax, Peanut, Rapeseed, Safflower, Sesame, Sunflower, Soybean, Root/Tuber and Corm Vegetables, Carrot, Potato, Sweet Potato, Beets, Ginger, Horseradish, Radish, Ginseng, Turnip, sugarcane, sugarbeet, Grass, or Turf grass.

22. The plant seed of claim 19, wherein the composition further comprises one or a combination of a microbial or a chemical insecticide, fungicide, nematicide, bacteriocide, herbicide, plant extract, or plant growth regulator present in an amount suitable to benefit plant growth.

23. The plant seed of claim 22, wherein the insecticide comprises bifenthrin.

24. The plant seed of claim 22, wherein the nematicide comprises cadusafos.

25. The plant seed of claim 22, wherein the insecticide comprises bifenthrin and clothianidin.

26. A composition for benefiting plant growth, the composition comprising:
a biologically pure culture of Bacillus licheniformis RTI184 deposited as ATCC No. PTA-121722, or a mutant thereof having all the identifying characteristics thereof, in an amount suitable to benefit plant growth; and
one or a combination of a microbial or a chemical insecticide, fungicide, nematicide, bacteriocide, herbicide, plant extract, or plant growth regulator, in an amount suitable to benefit plant growth.

27. The composition of claim 26, wherein the composition is in the form of a liquid or an oil dispersion and the Bacillus licheniformis RTI184 is present at a concentration of from about 1.0×10⁹ CFU/ml to about 1.0×10¹² CFU/ml.

28. The composition of claim 26, wherein the composition is in the form of a dust, a dry wettable powder, a spreadable granule, or a dry wettable granule and the Bacillus licheniformis RTI184 is present in an amount of from about 1.0×10⁹ CFU/g to about 1.0×10¹² CFU/g.

29. The composition of claim 26, wherein the Bacillus licheniformis RTI184 is in the form of spores.

30. The composition of claim 26, wherein the Bacillus licheniformis RTI184 is in the form of vegetative cells.

31. The composition of claim 26, wherein the plant comprises monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

32. The composition of claim 26, wherein the insecticide comprises bifenthrin.

33. The composition of claim 26, wherein the nematicide comprises cadusafos.

34. The composition of claim 26, wherein the insecticide comprises bifenthrin and clothianidin.

35. The composition of claim 26, formulated as a liquid.

36. The composition of claim 35, wherein the insecticide comprises bifenthrin or zeta-cypermethrin.

37. A method for benefiting growth of a plant, the method comprising delivering a composition comprising a biologically pure culture of Bacillus licheniformis RTI184 deposited as ATCC No. PTA-121722, or a mutant thereof having all the identifying characteristics thereof to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit plant growth.

38. The method of claim 37, wherein the growth benefit of the plant is exhibited by improved seedling vigor, improved root development, improved plant growth, improved plant health, increased yield, or improved appearance, or a combination thereof.

39. The method of claim 37, wherein the Bacillus licheniformis RTI184 is delivered at a rate of about 1.0×10⁸ CFU/ha to about 1.0×10¹³ CFU/ha.

40. The method of claim 37, wherein the Bacillus licheniformis RTI184 is in the form of spores.

41. The method of claim 37, wherein the Bacillus licheniformis RTI184 is in the form of vegetative cells.

42. The method of claim 37, wherein the composition is in the form of a liquid, an oil dispersion, a dust, a dry wettable powder, a spreadable granule, or a dry wettable granule.

43. The method of claim 37, wherein the plant comprises monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

44. The method of claim 37, wherein the composition further comprises one or a combination of a microbial or a chemical insecticide, fungicide, nematicide, bacteriocide, herbicide, plant extract, or plant growth regulator, present in an amount suitable to benefit plant growth and/or to confer protection against pathogenic infection in the susceptible plant.

45. The method of claim 44, wherein the insecticide comprises bifenthrin.

46. The method of claim 44, wherein the nematicide comprises cadusafos.

47. The method of claim 50, wherein the insecticide comprises bifenthrin and clothianidin.

48. The method of claim 44, formulated as a liquid.

49. The method of claim 48, wherein the insecticide comprises bifenthrin or zeta-cypermethrin.

50. A method for benefiting growth of a plant, the method comprising:
planting a seed of the plant or regenerating vegetative/callus tissue of the plant in a suitable growth medium, wherein the seed has been coated or the vegetative/callus tissue has been inoculated with a composition comprising a biologically pure culture of Bacillus licheniformis RTI184 deposited as ATCC PTA-121722, or a mutant thereof having all the identifying characteristics thereof, wherein growth of the plant from the seed or the vegetative/callus tissue is benefited.

51. The method of claim 50, wherein the growth benefit of the plant is exhibited by improved seedling vigor, improved root development, improved plant growth, improved plant health, increased yield, or improved appearance, or a combination thereof.

52. The method of claim 50, wherein the Bacillus licheniformis RTI184 is present in the form of spores at an amount of from about 1.0×10² CFU/seed to about 1.0×10⁹ CFU/seed.

53. The method of claim 50, wherein the seed comprises monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

54. The method of claim 50, wherein the composition further comprises one or a combination of a microbial or a chemical insecticide, fungicide, nematicide, bacteriocide, herbicide, plant extract, or plant growth present in an amount suitable to benefit plant growth.

55. The method of claim 54, wherein the insecticide comprises bifenthrin.

56. The method of claim 54, wherein the nematicide comprises cadusafos.

57. The method of claim 54, wherein the insecticide comprises bifenthrin and clothianidin.

58. The method of claim 54, formulated as a liquid.

59. The method of claim 58, wherein the insecticide comprises bifenthrin or zeta-cypermethrin.

60. The method of claim 50, wherein the Bacillus licheniformis RTI184 is in the form of spores.

61. The method of claim 50, wherein the Bacillus licheniformis RTI184 is in the form of vegetative cells.

62. A method for benefiting plant growth, the method comprising:
delivering a combination of:
a first composition comprising a biologically pure culture of Bacillus licheniformis RTI184 deposited as ATCC No. PTA-121722, or a mutant thereof having all the identifying characteristics thereof in an amount suitable for benefiting plant growth; and
a second composition comprising one or a combination of a microbial or a chemical insecticide, fungicide, nematicide, bacteriocide, herbicide, plant extract, or plant growth regulator, in an amount suitable for benefiting plant growth, to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

63. The method of claim 62, wherein the plant growth benefit is exhibited by improved seedling vigor, improved root development, improved plant health, increased plant mass, increased yield, improved appearance, or improved resistance to plant pathogens, or a combination thereof.

64. The method of claim 62, wherein the amount suitable for benefiting plant growth is from about 1.0×10⁸ CFU/ha to about 1.0×10¹³ CFU/ha Bacillus licheniformis RTI184.

65. The method of claim 62, wherein the Bacillus licheniformis RTI184 is in the form of spores.

66. The method of claim 62, wherein the Bacillus licheniformis RTI184 is in the form of vegetative cells.

67. The method of claim 62, wherein the plant comprises monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

68. The method of claim 62, wherein the insecticide comprises bifenthrin.

69. The method of claim 62, wherein the nematicide comprises cadusafos.

70. The method of claim 62, wherein the insecticide comprises bifenthrin and clothianidin.

71. The method of claim 62, wherein the insecticide is formulated as a liquid.

72. The method of claim 71, wherein the insecticide comprises bifenthrin or zeta-cypermethrin.

73. A method for benefiting plant growth, the method comprising:
delivering a composition comprising:
a biologically pure culture of Bacillus licheniformis RTI184 deposited as ATCC No. PTA-121722, or a mutant thereof having all the identifying characteristics thereof in an amount suitable for benefiting plant growth; and
one or a combination of a microbial or a chemical insecticide, fungicide, nematicide, bacteriocide, or plant growth regulator, in an amount suitable for benefiting plant growth,
to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

74. The method of claim 73, wherein the plant growth benefit is exhibited by improved seedling vigor, improved root development, improved plant health, increased plant mass, increased yield, improved appearance, or improved resistance to plant pathogens, or a combination thereof.

75. The method of claim 73, wherein the Bacillus licheniformis RTI184 is in the form of spores.

76. The method of claim 73, wherein the Bacillus licheniformis RTI184 is in the form of vegetative cells.

77. The method of claim 73, wherein the plant comprises monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

78. The method of claim 73, wherein the amount suitable for benefiting plant growth is from about 1.0×10⁸ CFU/ha to about fertilizer 1.0×10¹³ CFU/ha Bacillus licheniformis RTI184.

79. The method of claim 73, wherein the insecticide comprises bifenthrin.

80. The method of claim 73, wherein the nematicide comprises cadusafos.

81. The method of claim 73, wherein the insecticide comprises bifenthrin and clothianidin.

82. The method of claim 73, formulated as a liquid.

83. The method of claim 82, wherein the insecticide comprises bifenthrin or zeta-cypermethrin.

84. A method for benefiting plant rooting, the method comprising:
dipping a cutting of a plant in a composition and planting it in a suitable growth medium, wherein the composition comprises a biologically pure culture of a Bacillus licheniformis strain RTI184 deposited as ATCC PTA-121722, or a mutant thereof having all the identifying characteristics thereof, in an amount suitable to benefit plant rooting, wherein root formation and growth of the plant from the cutting is benefited.

85. The method of claim 84, wherein the Bacillus licheniformis RTI184 is in the form of spores.

86. The method of claim 84, wherein the Bacillus licheniformis RTI184 is in the form of vegetative cells.

87. The method of claim 84, wherein the composition is in the form of a liquid or a dry wettable powder.

88. The composition of claim 87, wherein the composition is in the form of a dry wettable powder and the Bacillus licheniformis RTI184 is present in an amount of from about 1.0×10⁷ CFU/g to about 1.0×10⁹ CFU/g.

89. A composition comprising at least one of an isolated Fengycin MB-Cit compound and an isolated Dehydroxyfengycin MB-Cit compound in an amount suitable to confer one or both of a growth benefit on a plant or protection against a pathogenic infection in a susceptible plant, the Fengycin MB-Cit and Dehydroxyfengycin MB-Cit compounds having the formula: 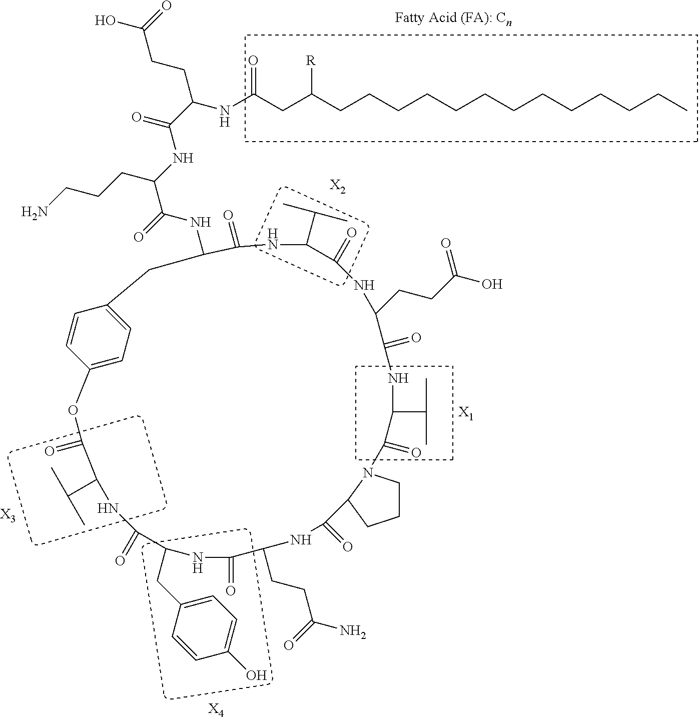
wherein n ranges from 8 to 20, FA is linear, iso, or anteiso, and R is OH, X₁ is Val, X₂ is Thr, X₃ is Met, and X₄ is Cit for Fengycin MB-Cit; and wherein n ranges from 8 to 20, FA is linear, iso, or anteiso, R is H, X₁ is Val, X₂ is Thr, X₃ is Met, and X₄ is Citruline for Dehydroxyfengycin MB-Cit.

90. The composition of claim 89, further comprising one or a combination of a microbial or a chemical insecticide, fungicide, nematicide, bacteriocide, herbicide, plant extract, or plant growth regulator, present in an amount suitable to benefit plant growth and/or to confer protection against pathogenic infection in the susceptible plant.

91. The composition of claim 89, wherein the composition is in the form of a liquid, a dust, a spreadable granule, or a dry wettable granule.

92. An extract of a biologically pure culture of a Bacillus licheniformis strain, the extract including at least one of a Fengycin MB-Cit compound and a Dehydroxyfengycin MB-Cit compound.

93. An extract of a biologically pure culture of Bacillus licheniformis RTI184 deposited as ATCC No. PTA-121722, the extract including a at least one of a Fengycin MB-Cit compound and a Dehydroxyfengycin MB-Cit compound.

94. A method for protecting or treating plants or fruit from a pathogenic infection, comprising applying an effective amount of the composition or the extract of any of claim 89, 90, 91, 92, or 93 to the plant or fruit, or to the roots or soil around the roots of the plants.

95. The method of claim 94, wherein the plant comprises monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hardwood cuttings, Chestnuts, Oak, Maple, sugarcane, or sugarbeet.

96. The method of claim 94, wherein the pathogenic infection is caused by a plant fungal pathogen, a plant bacterial pathogen, a rust fungus a Botrytis spp., a Botrytis cinerea, a Botrytis squamosa, an Erwinia spp., an Erwinia carotovora, an Erwinia amylovora, a Dickeya spp., a Dickeya dadantii, a Dickeya solani, an Agrobacterium spp., a Agrobacterium tumefaciens, a Xanthomonas spp., a Xanthomonas axonopodis, a Xanthomonas campestris pv. carotae, a Xanthomonas pruni, a Xanthomonas arboricola, a Xanthomonas oryzae pv. oryzae, a Xylella spp., a Xylella fastidiosa, a Candidatus spp., a Candidatus liberibacter, a Fusarium spp., a Fusarium graminearum, a Fusarium oxysporum, a Fusarium oxysporum f. sp. Cubense, a Sclerotinia spp., a Sclerotinia sclerotiorum, a Sclerotinia minor, Sclerotinia homeocarpa, a Cercospora/Cercosporidium spp., an Uncinula spp., an Uncinula necator (Powdery Mildew), a Podosphaera spp. (Powdery Mildew), a Podosphaera leucotricha, a Podosphaera clandestine, a Phomopsis spp., a Phomopsis viticola, an Alternaria spp., an Alternaria tenuissima, an Alternaria porri, an Alternaria alternate, an Alternaria solani, an Alternaria tenuis, a Pseudomonas spp., a Pseudomonas syringae pv. Tomato, a Phytophthora spp., a Phytophthora infestans, a Phytophthora parasitica, a Phytophthora sojae, a Phytophthora capsici, a Phytophthora cinnamon, a Phytophthora fragariae, a Phytophthora spp., a Phytophthora ramorum, a Phytophthora palmivara, a Phytophthora nicotianae, a Phakopsora spp., a Phakopsora pachyrhizi, a Phakopsora meibomiae an Aspergillus spp., an Aspergillus flavus, an Aspergillus niger, a Uromyces spp., a Uromyces appendiculatus, a Cladosporium spp., a Cladosporium herbarum, a Rhizopus spp., a Rhizopus arrhizus, a Penicillium spp., a Rhizoctonia spp., a Rhizoctonia solani, a Rhizoctonia zeae, a Rhizoctonia oryzae, a Rhizoctonia caritae, a Rhizoctonia cerealis, a Rhizoctonia crocorum, a Rhizoctonia fragariae, a Rhizoctonia ramicola, a Rhizoctonia rubi, a Rhizoctonia leguminicola, a Macrophomina phaseolina, a Magnaorthe oryzae, a Mycosphaerella spp., Mycosphaerella graminocola, a Mycosphaerella fijiensis (Black sigatoga), a Mycosphaerella pomi, a Mycosphaerella citri, a Magnaporthe spp., a Magnaporthe grisea, a Monilinia spp., a Monilinia fruticola, a Monilinia vacciniicorymbosi, a Monilinia laxa, a Colletotrichum spp., a Colletotrichum gloeosporiodes, a Colletotrichum acutatum, a Colletotrichum Candidum, a Diaporthe spp., a Diaporthe citri, a Corynespora spp., a Corynespora Cassiicola, a Gymnosporangium spp., a Gymnosporangium juniperi-virginianae, a Schizothyrium spp., a Schizothyrium pomi, a Gloeodes spp., a Gloeodes pomigena, a Botryosphaeria spp., a Botryosphaeria dothidea, a Neofabraea spp., a Wilsonomyces spp., a Wilsonomyces carpophilus, a Sphaerotheca spp., a Sphaerotheca macularis, a Sphaerotheca pannosa, a Erysiphe spp., a Stagonospora spp., a Stagonospora nodorum, a Pythium spp., a Pythium ultimum, a Pythium aphanidermatum, a Pythium irregularum, a Pythium ulosum, a Pythium lutriarium, a Pythium sylvatium, a Venturia spp, a Venturia inaequalis, a Verticillium spp., a Ustilago spp., a Ustilago nuda, a Ustilago maydis, a Ustilago scitaminea, a Claviceps spp., a Claviceps puprrea, a Tilletia spp., a Tilletia tritici, a Tilletia laevis, a Tilletia horrid, a Tilletia controversa, a Phoma spp., a Phoma glycinicola, a Phoma exigua, a Phoma lingam, a Cocliobolus sativus, a Gaeumanomyces gaminis, a Colleototricum spp., a Rhychosporium spp., Rhychosporium secalis, a Biopolaris spp., a Helminthosporium spp., a Helminthosporium secalis, a Helminthosporium maydis, a Helminthosporium solai, a Helminthosporium tritici-repentis, or combinations thereof.


SEQUENCE LISTING (2 sequences)

SEQ ID NO: 1 - DNA, 1545 bp, Bacillus licheniformis
agagtttgatcctggctcaggacgaacgctggcggcgtgcctaatacatgcaagtcgagcggacagatgggagcttgctccctgatgtcagcggcggacgggtgagtaacacgtgggtaacctgcctgtaagactgggataactccgggaaaccggggctaataccagatgcttgattgaaccgcatggttcaattataaaaggtggcttttagctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacaaggtagccgtatcggaaggtgcggctggatcacctcctttct

SEQ ID NO: 2 - DNA, 3015 bp, Bacillus licheniformis
ttgccggttacggttcttttgcgtgctcttggcttcggatctgaccaagagatcattgatctcatcggtgaaaacgagtatttgcgcaatacgcttgataaagataatacggaaaacaccgataaagcgcttcttgaaatctacgagcgtcttcgtccaggagagccgcctacagtagaaaacgcaaaaagcctgcttgattcaaggttctttgatccgaaaagatatgaccttgcaagtgtaggacgttataaaattaataaaaagcttcacatcaaaaacagactgtttaatcagcggcttgctgaaacgctgatcgatcctgaaacaggcgaaattcttgccgaaaaaggcgcgattttagacagaagaacgcttgacaaagtcctgccttaccttgaaaacggaatcggttttaaaaagctgtatccgaacggcggagttgttgaagatgaagtgacgcttcagtctattaaaatctatgccccgacagatcaagaaggggagcagacaatcaatgtgattggaaatgcttatattgaagaaggcgttaaaaatattacaccttctgatattatctcttccatcagctatttctttaacctgcttcacggagtgggcgataccgacgatatcgaccatttaggaaaccgccgtctccgttcagtgggagagcttctgcaaaaccaattccgtatcggtttaagcagaatggagcgtgttgttcgtgaaagaatgtctattcaagatacaaacacgatcacgccacagcagctgatcaatattcgccctgtcatcgcatcaatcaaagagtttttcggaagctcgcagctttctcagtttatggatcagacgaatccgcttgctgagctgacgcataagcgccgtctgtcagcgctcggaccgggcggtttgacccgtgagcgcgccggaatggaagtccgtgacgttcactattcccactacggccggatgtgtccgattgaaactcctgagggtccaaacatcggcttgatcaactcgctttcttcattcgcgaaagtgaaccgtttcggcttcatcgaaacgccgtatcgccgcgttgaccctgagactggaaaagtaacgccgagaatcgattacttgacagctgatgaagaagacaactatgtcgttgcccaggctaacgcacgtctgaatgatgacggatcttttgtggatgacagcatcgtcgcccgtttcagaggggagaacaccgttgttccgaaagaccgcgtcgactatatggacgtttcgcctaaacaggttgtctctgccgcgactgcatgtattcctttcttggaaaacgacgactcaaaccgcgccctaatgggagcgaacatgcaacggcaggctgtacctcttatgcagcctgaatctccgatcgtcggaaccggaatggagtatgtatctgcaaaagactccggtgccgctgttatttgccgccatcctggaatcgttgaacgggtggaagcgaaaaacatctgggtgcgccgctatgaagaagtcgacggccagaaagtcaaaggaaaccttgataaatacagcctgctgaagtttgttcgttccaaccagggaacttgctacaaccagcgtccgatcgtaagcgtcggtgatgaggttgtaaaaggtgaaattttagctgacggtccgtctatggaaaaaggtgagcttgcccttggacgcaacgtcatggtcggctttatgacatgggatggctacaactacgaggatgccatcatcatgagcgaacgccttgtaaaagacgacgtatacacgtctattcatattgaggaatacgaatcagaggcccgggatacaaaactcggacctgaagaaattacacgcgatattccaaacgtcggtgaagacgctcttcgcaatctcgatgaacgcggaattatccgtgtcggtgctgaagtaaaagacggagatcttcttgttggtaaagtaacgcctaaaggtgttacagagcttactgcagaagagcgcctgcttcacgccatcttcggcgaaaaagcgcgtgaagtccgcgatacgtcgctgcgtgtgcctcacggaggcggcggtatcatccttgatgtaaaagtgttcaaccgcgaagacggagacgaactgcctccgggcgttaaccagctcgtccgcgtttacatcgttcagaaacgtaaaatttctgaaggggacaaaatggccggacgccacgggaacaaaggtgttatttcgaaaatccttccggaggaagatatgccgtatctgcctgacggaacaccgattgacatcatgttaaacccgctgggcgtaccatcgcgtatgaacatcgggcaggtgttggagctgcaccttggtatggctgcacgccgtctcggtctgcatgtcgcgtcgccggtatttgacggtgcccgcgaagaagatgtgtgggaaacgcttgaagaagccggcatgtcaagggacgcaaagacagtcctttacgacggccgaactggagagccgtttgacaaccgggtgtctgtcggcatcatgtacatgatcaaattggctcacatggttgacgacaaattgcacgcgcgttctacaggtccttactcactcgttacccagcagcctcttggaggtaaagcgcagttcggtggacagcgttttggagagatggaagtttgggcgcttgaagcttacggtgcagcatatacacttcaagaaatcctgactgttaaatcggatgatgtcgtaggccgtgtgaaaacatacgaagccatcgtaaaaggcgacaatgttccagaacctggtgttccggaatcgttcaaagtattgatcaaagagcttcaaagcttaggtatggacgtcaaaatcctatcaagcgacgaagaagaaatcgaaatgagagacttggaagacgacgaagacgcgaaacaaaacgaagggctttctctgccgaatgatgaagagtccgaagaattcgtttctgctgacgcagagcgcgatgtcgtcacaaaagaataa